(19) United States
(12) Patent Application Publication
(10) Pub. No.: US 2023/0250428 A1
(43) Pub. Date: Aug. 10, 2023

(54) Method and composition for early detection of malignancies

(71) Applicant: Alliance of Cardiovascular Researchers, Houston, TX (US)

(72) Inventors: Eckhard U. ALT, Houston, TX (US); Reza IZADPANAH, Covington, LA (US); Amin IZADPANAH, Covington, LA (US)

(21) Appl. No.: 18/165,760

(22) Filed: Feb. 7, 2023

Related U.S. Application Data
(60) Provisional application No. 63/307,358, filed on Feb. 7, 2022.

(51) Int. Cl. C12N 15/113; C12N 15/63; A61P 35/04

(52) U.S. Cl. CPC C12N 15/113; C12N 15/63; A61P 35/04; C12N 2310/11

(57) ABSTRACT

A method and composition for treating a subject having a difficult to treat tumor is described. A TRAF3IP2 silencer is administered to the subject, preferably together with other synergistic medication such as chemotherapy, immunotherapies or other anticancer medication, that given alone are less or not successful for treating this special tumor, to increase relapse free survival. The TRAF3IP2 silencer in conjunction with other synergistic medication can additionally be used to effectively treat or prevent metastasis in a subject having an otherwise difficult to treat tumor.

### PRIOR RELATED APPLICATIONS

This invention claims priority to U.S. provisional application number 63/307,358, filed on Feb. 7, 2022 and incorporated by reference in its entirety herein for all purposes.

### FIELD OF THE DISCLOSURE

The disclosure generally relates to a method for synergistic treatment of difficult to treat malignant tumors, and more specifically to a method of not only detecting early malignancy of tumors by assessing the expression of tRAF3IP2, but to treat such otherwise difficult to treat malignancies by silencing TRAF3IP2 alone, or better in combination with additional medication that alone has demonstrated or is known to be less or not effective for treatment of those cancer.

### BACKGROUND OF THE DISCLOSURE

Triple-negative breast cancer (TNBC) is the most aggressive and deadliest subtype of breast cancer and has limited therapeutic options. The critical barrier to progress is the lack of targets that prevent and/or eliminate metastasis, impact on or block cancer stem cell (CSCs) function, to increase survival. Specifically, in the advanced metastatic stage, TNBC is managed by a combination chemotherapy, which is associated with very low response rates and poor survival benefit.

Analysis of data from The Cancer Genome Atlas (TCGA, accessed through GDC, GEPIA, and BreastMark) indicates an inverse correlation between TRAF3IP2 expression and disease-free survival (DFS) in TNBC patients (FIG. 1).

TRAF3IP2 increases pro-inflammatory signaling through activation of JNK, AP-1, and NF-υB. It is reported that this occurs in many cancers including TNBC and glioblastoma, and that TRAF3IP2 is a key regulator of the pro-tumorigenic Tumor Micro Environment (TME) in those malignant diseases.

### SUMMARY OF THE DISCLOSURE

In one aspect, a method of treating a subject having cancer, comprising the steps of: a) administering a pharmaceutical composition to the subject, and b) optionally along with additional synergistic measures such as chemotherapy, or with other immunotherapeutic, antiangiogenic medication or pharmaceutical composition to reduce the size of the tumor or eliminate it; c) wherein the pharmaceutical composition comprises an effective amount of at least one targeting sequence against at least a portion of TRAF3IP2 in a pharmaceutically acceptable carrier.

In another aspect of this disclosure, a composition for treating a subject having cancer is described. The composition comprises a therapeutically effective amount of a TRAF3IP2 silencer, and a pharmaceutically acceptable carrier, wherein the TRAF3IP2 silencer comprises at least one targeting sequence against at least a portion of TRAF3IP2 to silence expression of TRAF3IP2 in cancer cells.

### Primary Human TNBC Tumors Express High Levels of TRAF3IP2 (FIG. 2).

Using triple-negative breast cancer as an example, it is reported that TRAF3IP2 plays a critical role in metabolic reprogramming in TNBC. This disclosure also shows that silencing TRAF3IP2 inhibits mTOR (Mechanistic Target Of Rapamycin Kinase) and NAMPT (Nicotinamide Phosphoribosyltransferase), while inducing LKB1 (liver kinase B1) and AMPK (AMP-activated protein kinase).

When TRAF3IP2 expression is silenced in TNBC cells, senesence is induced without activation of the pro-inflammatory senescence-associated secretory phenotype (SASP) due to inhibition of pro-inflammatory cytokine signaling. This results in reduced cell proliferation and angiogenesis. In other words, silencing TRAF3IP2 inhibits angiogenesis, and a patient derived xenograft (PDX) model is used as an example.

TRAF3IP2 is a major regulator of cancer stem cells (CSCs), and suppresses key stem cell maintenance mechanisms, including inhibition of ATP binding cassette (ABC) transporters and Wnt/β-Catenin signaling and Sox2 levels. In one embodiment of this disclosure, it is shown that silencing TRAF3IP2 suppresses TNBC tumorigenesis, resulting in inhibition of tumor growth and metastasis.

Additionally, TRAF3IP2 silencer can have synergistic effect when administered with chemotherapy medication. In one embodiment, silencing TRAF3IP2 synergizes with paclitaxel and reverse chemoresistance in a chemoresistant TNBC PDX model.

For example, a short hairpin silencer (shRNA) generally has about 18-30 nucleotides (nt), preferably 21 nt, comprising a unique sense strand of target mRNA beginning with AA linked to a loop (3-9 nt) linked to an complement of the unique sense strand and finishing with polyT, thus forming a hairpin. An initiating G nt could also be used.

Another type of silencer is the siRNA of about 18-30 nt, preferably 21 nt, comprising a unique sense strand of the target mRNA beginning with AA and finishing with polyT.

Another type of silencer is the antisense sequence. These can be a unique antisense sequence from the target, or an RNAse resistant 18-30 nt antisense RNA sequence from the target. Effective antisense silencers may also be located in exons, but close to the acceptor splice site (SS).

miRNAs generally work when about 21-23 nt and have complementarity maintained in the first third of the small RNA and target mRNA, but mismatches arise in the remainder of the aligned sequence.

The above rules are guidelines only, however, and there is certainly variability in approaches. Therefore, it is typical to design 4-6 such silencers using the basic rules and then test each for activity, e.g., in an ex vivo system. Therefore, given the validity of the target, silencers can be readily be designed using the target sequence.

There are also non-viral methods of silencer delivery, including e.g, injecting naked DNA/RNA into a tumor, injected protected RNA into tumors, electrotransfection, the use of polymers, liposomes, and the like, to protect the nucleic acids, or to stabilize the silencer through linking it to Protamin.

An example of the vector is lentiviral vectors or parvovirus HP1 or LuIII. Lentiviral vectors were used herein to encode the silencer sequences for TRAF3IP2. Although data show that there is specificity for CD45+ cells transduction in vivo when administering lentiviral vectors, MDA-MB231 and SW620 cells are highly transducible with lentiviral vectors. However, any suitable expression vector may be used herein, or the gene can be introduced into the genome of a homing cell (e.g., by homologous recombination), such as the MSCs discussed herein.

Common vectors are based on herpes simplex type 1 recombinant vector (HSV-1); adenovirus, adeno-associated viral vector (AAV); alpha virus; vaccinia virus; pox virus; sendai virus; plasmids; retrovirus; ssDNA vectors; and the like. To date, adenovirus, retrovirus and naked plasmid DNA have made up more than half of the vectors tested in clinical trials of various gene therapies.

The disclosure provides one or more of the following embodiments, in any combinations(s) thereof:

--A method of treating a subject having cancer, comprising the steps
of: a) administering a pharmaceutical
composition to the subject, and b) optionally administering a
chemotherapeutic compound to the subject along with the pharmaceutical
composition; c) wherein the pharmaceutical
composition comprises an effective amount of at least one
targeting sequence against at least a portion of TRAF3IP2 in a
pharmaceutically acceptable carrier.
--Any method herein described, wherein the targeting
sequence against at least a portion of TRAF3IP2 is
selected from the group consisting of: a siRNA, a miRNA, a shRNA,
an antisense RNA, or an antisense oligonucleotide.
--Any method herein described, wherein the targeting sequence is
encoded by an expression vector,
wherein the targeting sequence is operably coupled to an inducible
promoter in the expression vector.
--Any method herein described, wherein the cancer is triple negative
breast cancer (TNBC).
--Any method herein described, wherein the chemotherapeutic
compound is selected from the group consisting
of Actinomycin, All-trans retinoic acid, Azacitidine,
Azathioprine, Bleomycin, Bortezomib, Carboplatin, Capecitabine,
Cisplatin, Chlorambucil, Cyclophosphamide,
Cytarabine, Daunorubicin, Docetaxel, Doxifluridine,
Doxorubicin, Epirubicin, Epothilone, Etoposide, Fluorouracil,
Gemcitabine, Hydroxyurea, Idarubicin,
Imatinib, Irinotecan, Mechlorethamine, Mercaptopurine,
Methotrexate, Mitoxantrone, Oxaliplatin, Paclitaxel,
Pemetrexed, Teniposide,
Tioguanine, Topotecan, Valrubicin, Vemurafenib, Vinblastine,
Vincristine, and Vindesine.
--Any method herein described, wherein the
subject's metastasis is reduced by at least
50% as compared to the same treatment without administering
the pharmaceutical composition.
--Any method herein described, wherein the
administering step comprises parenteral
administration, including injection into a tumor or its metastasis site
by transcutaneous, intraarterial, intraductal,
intravenous, intradermal, intramuscular, intraperitoneal, or
subcutaneous administration.
--Any method herein described, wherein Wnt/β-Catenin activation
is reduced or inhibited.
--Any method herein described, wherein Fzd8 expression in cancer cells is
downregulated by at least 50% or inhibited.
--Any method herein described, wherein E-Cadherin expression
is upregulated by at least 50%.
--Any method herein described, wherein tumor growth is inhibited.
--A composition for treating a subject having caner, comprising: a) a
therapeutically effective amount
of a TRAF3IP2 silencer; b) a pharmaceutically acceptable carrier; and
c) one or more anticancer medication, wherein the combination of
the TRAF3IP2 silencer and the anticancer
medication is at least 20% more effective in treating the
cancer than the anticancer medication alone; wherein the
TRAF3IP2 silencer comprises at least one
targeting sequence against at least a portion of TRAF3IP2 to silence
expression of TRAF3IP2 in cancer cells.
--Any composition herein described, wherein the cancer is
triple negative breast cancer
(TNBC), glioblastoma, pancreatic cancer or metastatic ovarian cancer.
--Any composition herein described, wherein the anticancer medication is
chemotherapeutic agent, immunotherapeutic agent, antiangiogenic
agent, or combinations thereof.
--A method of sensitizing a tumor prior to treating a patient with the
tumor, comprising the steps of: a)
administering a pharmaceutical composition into a subject before at least
one of the following procedures for treating the tumor: chemotherapy,
radiation therapy, immunotherapy or
targeted intervention, wherein the pharmaceutical composition
comprises at least one targeting sequence for TRAF3IP2 in a
pharmaceutically acceptable carrier in
an amount effective for sensitizing the tumor; and b) performing
the procedure for treating the tumor.
--Any method of sensitizing a tumor herein described, wherein the
cancer is TNBC.

As used herein, the term “expression vector” means a DNA or RNA into which a sequence of interest can be inserted that operably linked to a promoter such that the sequence will be transcribed or expressed from the promoter in the host cell/animal of interest. Thousands of such vectors are available. See e.g., Addgene.org which provides both a repository and a searchable database allowing vectors to be easily located and obtained from colleagues. See also Plasmid Information Database (PlasmID) and DNASU having over 191,000 plasmids. A collection of cloning vectors is also kept at the National Institute of Genetics as a resource for the biological research community. Furthermore, vectors (including particular ORFS therein) are usually available from colleagues.

As used herein, the term “increased TRAF3IP2 expression” refers to the level of TRAF3IP2 in a cancer cell being higher than a normal cell. In one embodiment, it refers to at least 20% increase of the level of TRAF3IP2. In another embodiment, it refers to at least 30%, 40%, 50%, 100%, 200% or 100% increase of the level of TRAF3IP2 comparing to a normal cell.

As used herein, the term “targeting” refers to a nucleic acid sequence specifically complementary to and hybridize with at least a portion of a target gene.

As used herein, the term “silencing” refers to the down-regulation of gene expression. At least 65%, 70%, 75%, 80% reduction should be achieved, but preferably, this term refers to the ability of a cell to prevent the expression of a certain gene. Gene silencing can occur during either transcription or translation and is often used in research and gene therapies.

As used herein, the term “suppressing” refers to down-regulating gene expression by at least 30%, and preferably at least 40%, 45%, 50%, 55% or 60% down-regulation should be achieved.

By “preventing” gene expression, we mean no detectable intact gene expression is detected when assayed by Northern blot using a radioactively end-labeled oligomer that is complementary to the gene being silenced. Nonetheless, there may be minute amounts of expression that could be detected by extremely sensitive methods.

The term “silencer” as used herein refers to an exogenous sequence that can be introduced into cells and used to silence gene expression in that cell. There are several different types of silencers, including at least antisense oligonucleotides, ribozymes, RNA interference, and the like. Genes can be silenced by e.g., dsRNA that decomposes mRNA, siRNA molecules that cause the endonucleatic cleavage of the target mRNA molecules or by miRNA molecules that suppress translation of the mRNA molecule or by shRNA, as well as by endoribonuclease-prepared siRNAs (esiRNAs), which are a mixture of siRNA oligos resulting from cleavage of long double-stranded RNA (dsRNA) with an endoribonuclease such as Escherichia coli RNase III or dicer. The term “silencer” is not limited to any one particular methodology, unless so specified.

The term “inhibit” or inhibition refers to the down-regulation of the expression of a target gene or the reduction of a phenotype, by at least 10%, or at least 33%, or at least 50%, or at least 80% of its gene expression relative the control.

The term “sensitizing” refers to the synergistic induction of apoptosis in tumor cells by combining different therapies. For example, by modulating the DNA repair pathways inside the tumor cells, it is possible to sensitize cancer cells.

As used herein, the terms “operably associated,” “operably coupled” or “operably linked,” as used herein, refer to functionally coupled nucleic acid sequences.

As used herein, “pharmaceutically acceptable carrier” refers to any carrier that is capable of delivering oligonucleotide to target cells. Examples of the pharmaceutically acceptable carrier include, but not limited to, nucleic acid carrier, cationic lipids, peptide-mediated carrier such as cell-penetrating peptides, nanogel carrier, liposomes, small molecule tags (including cholesterol-modification, membrane-permeant peptides, folate, antibiotics, VITE, and VITA), and cationic polymers.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention.

The following abbreviations are used herein:

| ABBREVIATION | TERM |
|---|---|
| ABC | ATP-binding cassette |
| AMPK | AMP-activated protein kinase |
| AO | Antisense oligonucleotides |
| Cancer stem cells | CSC |
| DFS | Disease-free survival |
| ECM | Extracellular matrix |
| LKB1 | Liver kinase B1 |
| MRP1 | Multi-drug resistant protein 1) |
| mTOR | Mechanistic target of rapamycin kinase |
| NAMPT | Nicotinamide phosphoribosyltransferase |
| OS | Overall survival |
| PARP | poly (ADP-ribose) polymerase |
| PDX | Patient derived xenograft |
| RT-qPCR | real time quantitative PCR |
| SASP | Senescence-associated secretory phenotype |
| SLPI | Secretory leukocyte peptidase inhibitor |
| TME | Tumor micro environment |
| TNBC | Triple-negative breast cancer |

### BRIEF DESCRIPTION OF THE DRAWINGS

FIG. 1A. High levels of TRAF3IP2 are selectively associated with decreased disease-free survival (DFS) in TNBC. Analysis of preexisting data from GDC, GEPIA, and BreastMark showed a hazard ratio of 1.3 for higher, but not lower, TRAF3IP2 expression, indicating that higher TRAF3IP2 expression is associated with significantly lower DFS in TNBC. DFS and hazard ratio were calculated using Cox Proportional-Hazards model across TNBC cases; n=535 high and 535 low TRAF3IP2 in TNBC cases (HR=1.34, P=0.05).

FIG. 1B-1C. TRAF3IP2 overexpression in TNBC and predicts poor survival in basal-like breast cancer. FIG. 1B: Analysis of pre-existing TCGA data showed TNBC has significantly increased TRAF3IP2 expression compared to nontriple negative BC(p<0.0009). FIG. 1C: There is a hazard ratio of 1.37 for higher, but not lower, TRAF3IP2 expression (p=0.0043), indicating that higher TRAF3IP2 expression is associated with significantly lower OS in basal like BC, including TNBC. OS and hazard ratio were calculated using Cox Proportional-Hazards model across TNBC cases; n=403 low and 461 high TRAF3IP2. Data accessed through bc-GenExMiner on Feb. 1, 2023.

FIG. 2. TRAF3IP2 expression is increased in human primary TNBC tissues. Analysis of primary TNBC tumor tissues resected from patients revealed increased, but variable, TRAF3IP2 expression in four different primary TNBC tissue as shown by immunohistochemistry (IHC; brown), counterstained with hematoxylin (blue).

FIG. 3. Silencing TRAF3IP2 inhibits malignant metabolism in TNBC cells. Analysis of glycolysis in 4IC_{TRAF3IP2 KD} vs. 4IC_{control shRNA} cells (A). Seahorse assay showed silencing TRAF3IP2 significant changes in TNBC metabolism following targeting TRAF3IP2 (B). Membrane potential assay shows TRAF3IP2 silencing decreases membrane potential, indicating mitochondrial damage (C). Notably, the addition of FCCP, a potent uncoupler of mitochondrial oxidative phosphorylation, does not decrease mitochondrial membrane potential, suggesting that targeting TRAF3IP2 effectively reduces membrane potential and induces mitochondrial dysfunction.

FIG. 4. Silencing TRAF3IP2 increases LKB1 and AMPK and inhibits cell proliferation. Western blot showing upregulation of phosphorylated LKB1 (p-LKB1) and AMPK (p-AMPK) in 4IC_{TRAF3IP2 KD} vs. 4IC_{control shRNA} cells.

FIG. 5. Effect of TRAF3IP2 on NAMPT. Targeting TRAF3IP2 reduces levels of NAMPT in 4IC cells (p<0.01).

FIG. 6. Effect of targeting TRAF3IP2 on TNBC cancer stem cells. Once cultured in serum free media to isolate CSCs, the 4ICTRAF3IP2KD cells showed a significant impaired ability to form spheroids, while 4ICControl cells formed larger spheroids at 48 and 96 hours in culture (A&B). Flow cytometry showed a significant reduction of CD44 cells and increased CD24 cells in 4ICTRAF3IP2KD spheroids (C). Transcriptomic analysis showed a downregulation of inflammatory and angiogenic factors and increased in apoptosis marker (D).

FIG. 7. Targeting TRAF3IP2 enhances the efficacy of Paclitaxel in TNBC, resulting in cell cycle arrest. Cell cycle profiles of 4ICControl shRNA, 4ICControl shRNA+Paclitaxel, 4IC_{TRAF3IP2 KD} and 4IC_{TRAF3IP2 KD}+Paclitaxel (A-D). In Paclitaxel treatment alone, there was increase in the proportion of cells in G2 by 23%, as compared to a 12% increase in 4IC_{TRAF3IP2 KD}. Combined Paclitaxel administration and TRAF3IP2 silencing completely prevented entry into S phase and arrested cells in the G2/M checkpoint.

FIG. 8A-C. Targeting TRAF3IP2 significantly reduces levels of ABC Transporters (p<0.05).

FIG. 9. Targeting TRAF3IP2 significantly inhibits Wnt signaling through downregulating FZD8 and upregulating CDH1, evidenced by downregulation of c-Myc and CDK1. (p<0.05)

FIG. 10. Silencing TRAF3IP2 suppresses TNBC tumorigenesis. Female 4- to 6-weeks-old NSG mice were injected intramammary with 4IC_{TRAF}3IP2 kD cells (1×10⁶ cells in PBS and Matrigel) and compared to the control group (4IC_{Control shRNA})(n=10/group). Animals in the control group were euthanatized 4-weeks post inoculation due to large tumor growth (I). Significant gross metastasis was found in these animals in various tissues. The extent of metastasis in the abdomen, liver, kidneys and lungs is shown (arrows) (I). In contrast, 4IC_{TRAF3IP2 KD}-injected animals showed minimal tumor growth upon euthanasia after week 52 (II). Further, no metastasis was detected in major tissues.

FIG. 11. Effect of targeting TRAF3IP2 on metastasis. Targeting TRAF3IP2 significantly reduces genes critical in metastasis (p<0.05).

FIG. 12. Effect of targeting TRAF3IP2 on TNBC growth. A) 4IC tumors were induced in mammary fat pad of NSG mice. Treatment was initiated once measurable tumors were detected (day 14). Animals were treated with TRAF3IP2-AO (100 μg in 50 μl), administered subcutaneously in the vicinity of the tumors every 48 hours. Control group received SCRAO. Tumor volume was measured by calipers (primary tumor was unlabeled) every 48 hours prior to injection (n=5/group, *p<0.05 and **p<0.01). The image comparison of tumors treated with TRAF3IP2-AO and SCR-AO at day 28 post-injection. Size comparison of tumors shows smaller size and less vascularized tumors of TRAF3IP2-AO-treated. The study was terminated at day 28 post-injection. B) Comparative analysis of tumor mass was performed after harvesting. Red plot: Tumors from TRAF3IP2-AO-treated; Blue: Tumors from SCRAO- treated group (*p<0.01).

FIG. 13. Molecular analysis of targeting TRAF3IP2 in TNBC. A) RT-qPCR revealed the expression of genes related to inflammation, angiogenesis, cell cycle and apoptosis, and exosome release in tumors of TRAF3IP2-AO treated animals (vs. SCR-AO). B) Western Blotting Analysis of tumor lysates demonstrates significantly reduced levels of pro-tumorigenic proteins including reduced phosphorylated mTORC1 and mTORC2 in animals treated with TRAF3IP2-AO. C) Tumor tissue sections were stained with DAPI(blue-to show the cellular content), human specific mitochondria (redindicating the human/TNBC cells) and CD31 cells (purple-indicating blood vessels), scale bar 100 μm. Yellow arrows show decreased blood vessels in treated tumors.

FIG. 14. Detection of micrometastasis using PCR. Key TNBC metastatic niches were analyzed for micrometastasis using primers directed towards a humanspecific α-satellite DNA sequence of the centromere region of human chromosome 17. No human specific DNA sequence was detected in the most common metastatic sites in TRAF3IP2-AO treated animals.

FIG. 15. Treatment of late stage metastatic TNBC. 4IC tumors were induced intramammary into mice using 4IC cells [carrying a luciferase-expressing gene for ‘bioluminescence imaging’. Treatment was initiated in late stage BC indicated by advanced metastasis detectable by luciferase imaging (day 42). Animals were treated with TRAF3IP2-AO (100 m in 50 μl ) administered interperitoneally every 48 hours. Control group received SCR-AO in the same fashion (Data not shown). Representative images showing the extend of metastasis after treatment 1(T1), T5, T10, and T15. The control animals were treated with SCR-AO only on T1 and were euthanized due to the increasing tumor growth.

FIG. 16. Effect of targeting TRAF3IP2 on chemoresistance in vivo. Tumors were induced using 4IC cells (n=6 animals/group), and mice were treated with one of the following: SCR-AO, SCR-AO+Paclitaxel, TRAF3IP2-AO, or TRAF3IP2-AO+Paclitaxel. Animals in the 4IC SCR-AO group were euthanized by day 28 due to increased tumor n of 1600 mm³. Paclitaxel reduced tumor volume by 42% and TRAF3IP2 targeting reduced tumor size by 78%. Combination treatment eliminated the tumor size (p<0.05).

### DETAILED DESCRIPTION

The key innovation of our proposal is the identification of TRAF3IP2 as a key player in TNBC tumorigenesis, metabolic reprogramming, cancer stem cell formation and function, chemoresistance, and metastasis.

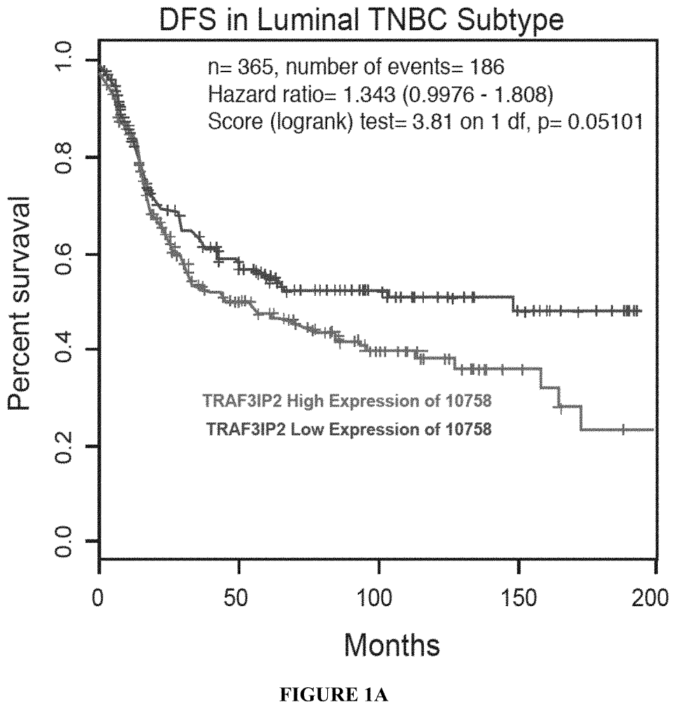

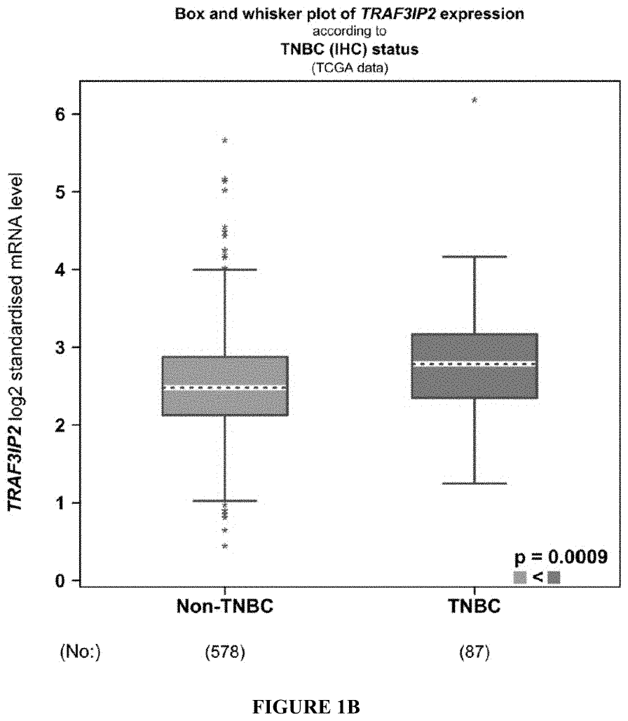

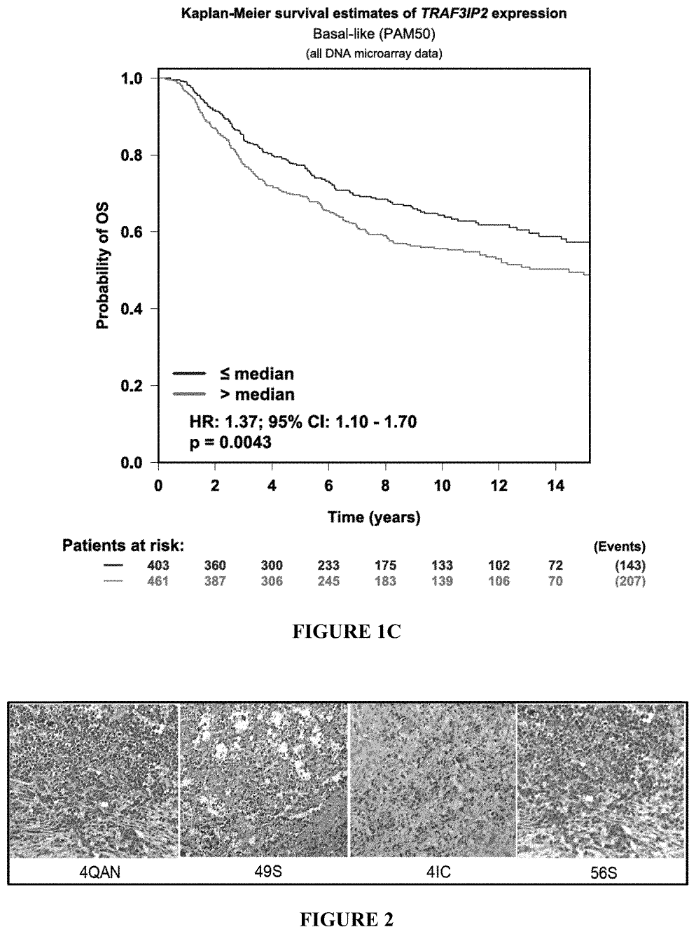

Triple-negative breast cancer (TNBC) is the most aggressive and deadliest subtype of breast cancers with limited therapeutic options. A critical barrier to its successful treatment is the lack of targets that prevent or eliminate metastasis, block the function of cancer stem-like cells (CSCs), and increase overall survival (OS). Specifically, at an advanced metastatic stage, though TNBC is managed by a combination of chemotherapeutics, it is associated with low response rates and poor OS. Analysis of TCGA data indicates an inverse correlation between TRAF3IP2 expression and overall survival in TNBC (basal-like patients, as shown in FIG. 1B and 1C.

Our experimental approach utilizes clinically relevant patient derived TNBC xenografts for accurately recapitulating early and metastatic disease to test the hypothesis that targeting TRAF3IP2 effectively inhibits TNBC, and TRAF3IP2 silencing is synergistic with current chemotherapies.

Methodologically, we are using advanced bioluminescence imaging to monitor disease (tumor size and macro and micrometastases in real time).

For detection of micrometastasis, we are utilizing a validated, highly sensitive PCR-based approach to detect human cancer cells in a background of mouse tissue.

Our targeting methodology utilizing antisense oligonucleotides (AO) is already optimized and validated, and is an FDA-approved approved treatment modality.

If successful, our approach utilizing four distinct heterogenous patient-derived TNBC specimens will identify TRAF3IP2 as a critical driver of TNBC growth and metastasis and potentially become a targeted therapy for TNBC, which is a major gap in the field.

Innovations include the novel role of TRAF3IP2 in: i) metabolic reprogramming via coordinating glycolysis, mitochondrial metabolism, and NAD metabolism and how this influences cell proliferation and angiogenesis and ii) CSC formation and function and how this influences tumorigenesis, chemoresistance, and metastasis.

The inventors i) delineate the role of TRAF3IP2 in driving TNBC pathogenesis using PDX models, ii) demonstrate the utility of targeting TRAF3IP2 in inhibiting cancer stem cells (CSCs), and iii) demonstrate that targeting TRAF3IP2 extends overall survival, decreases metastasis, and reduces chemoresistance in TNBC PDXs.

Based on our current data, this disclosure utilizes four biologically distinct patient-derived TNBCs to demonstrate the causal role of TRAF3IP2 in TNBC pathology, thereby addressing TNBC heterogeneity. Establishing initial proof-of-concept by demonstrating the causal role of TRAF3IP2 in TNBC tumorigenesis, we determined the efficacy of silencing TRAF3IP2 using TRAF3IP2-shRNA in a panel of TNBC PDXs established at the Tulane University School of Medicine (TU-SOM) PDX core (i.e., Tu-BcX-4IC, Tu-BcX-4QAN, TU-BcX-49S, and TU-BcX-56S).

TABLE 1
TNBC specimens are derived from patients that were diagnosed with TNBC. Epidemiologic characteristics
and pathologic stage are shown in Table, and demonstrate heterogeneity in the TNBC specimens

| | | | | | | Size | Nodal | |
|---|---|---|---|---|---|---|---|---|
| Specimen | ER | PR | HER2 | Race | Age | (T) | status (N) | Metastasis |
| 49S | Negative | Negative | Negative | Black | 36 | pT3 | pN2a | pMX |
| 4IC | Negative | Negative | Negative | White | 57 | pT4b | pN1a | pM1 |
| 56S | Negative | Negative | Negative | Black | 60 | pT2 | pN1 | pMX |
| 4QAN | Negative | Negative | Negative | Black | 79 | pT4b | pN3a | pM1 |

These specimens represent four distinct TNBC xenografts established from neo-adjuvant therapy-treated patients that exhibited limited response to chemotherapy. Immunohistochemistry confirmed the increased expression of TRAF3IP2 in all TNBC specimens (FIG. 2). Cells from each of the TNBC PDXs will be silenced for TRAF3IP2 using an already validated and optimized lentiviral vector carrying TRAF3IP2 shRNA, generating TNBC_{TRAF3IP2KD} cells (KD: knockdown). TNBC PDX cells treated with a nonspecific shRNA, TNBC_{Control}, will serve as controls. Downregulation of TRAF3IP2 will be determined by real time quantitative PCR (RT-qPCR) and confirmed by Western Blotting. These experiments occur 72 hours post transfection of TNBC PDX cells with the lentiviral vector. Cells from the 4IC PDX were used to generate the presented current data. In all experiments, the expression of TRAF3IP2 will be restored in TNBC_{TRAF3IP2 KD} cells (TNBC_{TRAF3IP2Restored} cells) using a TRAF3IP2 expressing lentiviral vector. This will further elucidate the causal role of TRAF3IP2 in TNBC tumorigenesis. It is anticipated that restoring TRAF3IP2 may re-enable tumorigenicity. All Tumors are induced by injecting cells into mammary fat pad of female NSG mice (1×106 cells suspended in 100 μl of PBS+Matrigel; n=10/group).

### EXAMPLE 1: DETERMINING TRAF3IP2 IN MALIGNANT METABOLIC REPROGRAMMING THAT MEDIATES CELL CYCLE PROGRESSION AND ANGIOGENESIS IN TNBC

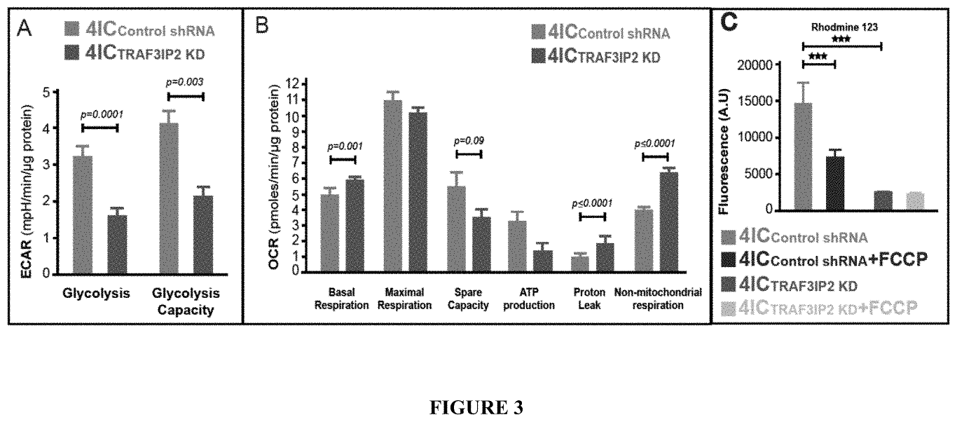

The key pro-tumorigenic features include malignant metabolism, neoangiogenesis, sustained cell proliferation, and inflammation. Tumor cells preferentially utilize glycolysis, rather than mitochondrial oxidative phosphorylation to generate and divert metabolic precursors for anabolic processes. These cells also generate large amounts of lactate (necessary to regenerate NAD+ for continued glycolytic activity) for several protumor growth functions, namely the acidification of the local microenvironment, which enhances tumor invasion and metastasis. Our current data conducted on cells derived from a TNBC patient (4IC cells) (26), demonstrate that silencing TRAF3IP2 induces significant metabolic effects (FIG. 3). Utilizing the Seahorse Analyzer, we demonstrated that targeting TRAF3IP2 in 4IC cells (4IC_{TRAF3IP2KD} cells) resulted in i) increased basal respiration, ii) increased proton leak, iii) decreased ATP production, and iv) increased non-mitochondrial respiration, compared to 4IC_{control shRNA} cells (FIG. 3(B)). The increase in non-mitochondrial respiration indicates continued tumor cell oxygen consumption without corresponding ATP production. To link this observation with the lack of ATP production, despite oxygen usage, we investigated the integrity of the mitochondrial electron transport chain and proton gradient; we observed a significant decrease in membrane potential of 4IC_{TRAF3IP2KD} cells resulting in inhibition of mitochondrial function (FIG. 3(C)). Notably, the addition of FCCP (an uncoupling agent) to 4IC_{TRAF3IP2KD} cells did not decrease membrane potential anymore, indicating that silencing of TRAF3IP2 is maximally effective in decreasing membrane potential. Therefore, our current understanding of the metabolic effects of silencing TRAF3IP2 is that targeting TRAF3IP2 promotes mitochondrial damage, evidenced by the dissipation of the mitochondrial membrane potential, resulting in cellular depletion of ATP. Further, we have evidence that glycolytic capacity in 4IC_{TRAF3IP2KD} cells is significantly reduced compared to 4IC_{control shRNA} (FIG. 3(A)). This finding is partially explained by the lack of reoxidation of NADH by the mitochondrial electron transport chain, which prevents glycolysis.

### 1. Effect of TRAF3IP2 on Metabolic Reprograming Primary TNBCs

The energy deficient state induced by targeting TRAF3IP2 is consistent with decreased tumor cell proliferation. Additionally, silencing TRAF3IP2 inhibits induction of NF-kB and significantly reduces the pro-inflammatory and pro-angiogenic cytokines IL1α, IL1β, IL6, and IL8. Here, we test the hypothesis that silencing TRAF3IP2 in TNBC PDXs induces metabolic injury that results in decreased tumor cell proliferation and angiogenesis. Studies in this Aim are performed on cells dissociated from authenticated patient derived TNBC specimens (listed above).

Cells from each of the TNBC PDXs are analyzed using Agilent Seahorse XF Real-Time ATP Rate Assay. TNBC_{TRAF3IP2KD} cells are compared with TNBC_{control} to determine the impact of targeting TRAF3IP2. In real time, the assay quantifies production of ATP from OXPHOS (oxidative phosphorylation) and glycolysis, separately, revealing the metabolic source of ATP in the cell. We then perform the Agilent Seahorse XF Cell Mito Stress Test, which determines mitochondrial function in the tumor cells. By measuring ATP-linked respiration, proton leak, maximal respiration, and spare respiratory capacity in TNBC_{TRAF3IP2KD} vs. TNBC_{control} cells, the assay reveals the metabolic consequences of targeting TRAF3IP2 in TNBC PDX cells.

It is then determined basal glycolytic activity and perform a glycolysis stress test to shift metabolism toward higher glycolytic activity to determine glycolytic reserve and capacity in TNBC_{TRAF3IP2KD} vs. TNBC_{control} cells. To determine and show whether silencing TRAF3IP2 induces alterations in cellular energy substrates, we investigated changes in glucose, pyruvate, fatty acid, and glutamine usage (Seahorse Substrate Oxidation Stress Test and Mito Fuel Flex Test).

### 2. Effect of Targeting TRAF3IP2 on Cell Proliferation and Angiogenesis

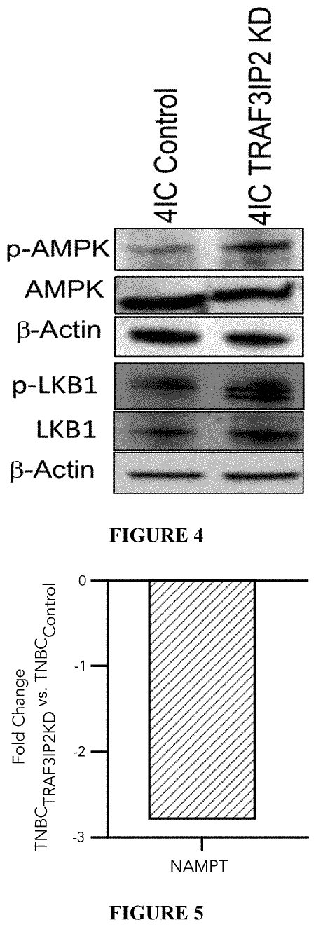

Our data show a significant upregulation of phosphorylated AMPK in 4IC_{TRAF3IP2 KD} cells (FIG. 4). LKB1 acts a master upstream kinase, directly phosphorylating and activating AMP-activated protein kinase (AMPK). The LKB1-AMPK pathway serves as a metabolic checkpoint in the cell, arresting cell growth in conditions of low intracellular ATP levels, such as in low nutrient conditions. Our data shows that phosphorylation of both LKB1 and AMPK increases in response to high AMP:ATP and ADP:ATP ratios in TRAF3IP2 silenced TNBC cells, signifying an energy deficient state. AMPK then results in a shift in the metabolic activity of the cell to favor OXPHOS, rather than the rapid glycolytic flux that tumor cells utilize during proliferation. AMPK activation has been shown to induce cell cycle arrest through activation of the p53/p21 axis. AMPK activation also results in downstream suppression of mTOR, resulting in decreased protein synthesis and growth.

Further, since mTOR activates HIF-la to induce VEGF expression, we anticipate that targeting TRAF3IP2 also suppresses VEGF resulting in reduced angiogenesis. Therefore, we hypothesize that targeting TRAF3IP2 induces metabolic injury in cancer cells through mitochondrial dysfunction and decrease in glycolytic flux, resulting in increased AMP:ATP ratio which will activate AMPK. Activated AMPK will induce cell cycle arrest, decreased protein synthesis and decreased VEGF expression via suppression of mTOR.

All experiments are performed in TNBC_{TRAF3IP2KD} vs. TNBC_{control} cells. Activation of AMPK will be investigated through Western blot using antibody against phospho-AMPK (activated) and total AMPK. Additionally, RT-qPCR will be utilized to determine expression differences. The serine/threonine kinase LKB1 phosphorylates Thr172 in the activation loop of AMPK, a reaction necessary for AMPK activation. Therefore, we will measure levels of LKB1 by RT-qPCR and Western blot. Activated mTOR (phospho-mTOR) and total mTOR and VEGF levels will be quantified by Western blot; expression levels of VEGF and mTOR will also be quantified using RT-qPCR. MTT assay will be performed to determine cell viability. Phosphoproteomic assays will be utilized to elucidate the effect of targeting TRAF3IP2 on kinase (MAPK) signaling, which drives cell cycle in cancer (Human Phospho-MAPK Array Kit, Biotechne). Cell cycle analysis will be performed to determine the proportion of cells in various stages of the cell cycle (G1, S, G2, M) and to identify cell cycle arrest.

It is anticipated to have consistently decreased production of ATP from both OXPHOS and glycolysis in TNBC_{TRAF3IP2KD} cells. We also anticipate decreased ATP-linked respiration and increased proton leak in TNBC_{TRAF3IP2KD} cells, consistent with metabolic injury. In TNBC_{TRAF3IP2KD} cells, we anticipate a decreased glycolytic reserve and capacity compared to TNBC_{control} cells. In TNBC_{TRAF3IP2KD} cells, a decrease in usage of glucose, fatty acid, and glutamine is anticipated; however, a disproportionately larger decrease in glucose dependence is more likely. It is expected that targeting TRAF3IP2 will activate AMPK; however, the predicted increase in OXPHOS will be blunted due to mitochondrial injury. We anticipate increased levels of LKB1, activated AMPK, and possibly increased total expression levels of AMPK, in TRAF3IP2-silenced cells vs. control cells. We predict decreased activation and levels of mTOR and VEGF. Finally, we anticipate these molecular changes induced by targeting TRAF3IP2 will result in decreased cell proliferation (decreased cell viability) and cell cycle arrest, evidenced by increased cell proportion in G1 and G2 phases and decreased cell proportion in S phase and M phase. This would suggest cell cycle arrest at G1/S and G2/M restriction points.

Based on the current data, targeting TRAF3IP2 in heterogenous TNBC samples consistently suppresses pro-tumorigenic metabolism, cell cycle progression, and angiogenesis. Moreover, silencing TRAF3IP2 significantly decreases glycolytic and mitochondrial function, decreases mTOR activation, and causes ATP deficiency and increase AMPK in all tested TNBC samples. Targeting TRAF3IP2 in heterogeneous TNBC consistently inhibits MAPK signaling and decreases pro-inflammatory cytokine signaling by targeting TRAF3IP2 downstream signaling. We also anticipate that TRAF3IP2 silencing will decrease VEGF expression.

### 3. TRAF3IP2 is Associated with NAMPT, Contributing to Metabolic Reprogramming. Silencing TRAF3IP2 Results in Inhibition of NAMPT and Induces SASP

NAMPT is the rate-limiting biosynthetic enzyme of the NAD+ salvage pathway, which converts nicotinamide to nicotinamide mononucleotide, a precursor to NAD+. Therefore, as a key regulator of the cellular NAD+ pool, NAMPT plays a critical role in cellular energetic homeostasis. BC cells have increased turnover of NAD+, and depend on NAMPT for regeneration of NAD+ for glycolysis and OXPHOS. Inhibiting NAMPT in MCF7 cell line and MDA-MB231 reduced NAD+ levels, reduced cell viability, and induced apoptosis. Importantly, the combination of NAMPT inhibition and PARP inhibitors demonstrated greater efficacy than either treatment alone in an in vivo TNBC model. Interestingly, cells treated with this combination demonstrated increased apoptotic markers, suggesting that NAMPT increased the lethality of PARP inhibitors to induce DNA damage and trigger apoptosis.

A major result of commonly used chemotherapeutics, including doxorubicin, cisplatin, methotrexate, taxanes, and PARP inhibitors, is the induction of cellular senescence to decrease tumor growth and metastasis. Despite an anti-proliferative phenotype, senescence is typically accompanied by a pro-inflammatory senescence associated secretory phenotype (SASP) involving the secretion of various cytokines including IL-6, IL-8, and IL-1b. The SASP has been associated with a pro-tumorigenic function and acquisition and enrichment of cancer stemness. Post-chemotherapy, spontaneous reemergence of enriched “stem-like” cells accounts of tumor recurrence and chemoresistance, both significant clinical challenges in the management of TNBC (39-42). Additionally, activation of the canonical Wnt/β-Catenin pathway has been implicated in the generation of therapy-induced, senescence associated CSCs (41) (We describe and propose the role of TRAF3IP2 in Wnt signaling in more detail in Specific Aim 2.2.). In ovarian cancer, NAMPT inhibition caused suppression of cisplatin induced, senescence-associated CSCs, a major cause of chemoresistance and tumor recurrence. Additionally, the inhibition of NAMPT has been linked with the induction of cellular senescence. NAMPT activity has broadly been associated with the pro-inflammatory and pro-tumorigenic SASP. Therefore, an ideal therapeutic strategy is to induce tumor cell senescence and eliminate the pro-tumorigenic SASP through downregulation of NAMPT, ultimately decreasing enrichment of CSCs and preventing development of chemoresistance and tumor recurrence.

Our current data indicate that targeting TRAF3IP2 induces mitochondrial dysfunction, partly through disruption of the mitochondrial membrane potential. This results in impairment of the electron transport chain and prevents reoxidation of reduced carriers, namely NADH and FADH2, thus generating an intracellular lack of NAD+ and impairment of glycolysis. Our data also indicate that targeting TRAF3IP2 reduces levels of NAMPT (FIG. 5), thus preventing the cancer cell from a feedback increase in NAD+ levels. We hypothesize that, within the cancer cell, the ensuing severe NAD+ deficiency contributes to further metabolic depression and initiates cell death or senescence. Our current data demonstrate TRAF3IP2 knockdown reduces cancer cell proliferation with concomitant reduction in secretion of pro-inflammatory cytokines such as IL-6, IL-8, and IL-1b. We therefore hypothesize that targeting TRAF3IP2, via inhibition of NAMPT, will induce metabolic dysfunction and senescence, without a pro-inflammatory SASP. We further hypothesize that the result of this is suppressed development of CSCs and chemoresistance.

To analyze the effects of targeting TRAF3IP2 on NAMPT, NAD+/NADH levels and ratios will be studied in TNBC_{TRAF3IP2KD} and compared to TNBC_{Control} cells from each of the TNBC PDXs (Table 1) (NAD/NADH-Glo™, Promega). Cell lysates will be used in a colorimetric NAMPT activity assay to detect NAMPT activity; we anticipate TNBC_{TRAF3IP2KD} will display lower NAMPT activity levels compared to TNBC_{Control} cells (NAMPT Activity Assay, colorimetric, Abcam, USA). Additionally, levels of NAMPT will be analyzed by RT-qPCR and Western blot. Upon confirmation of reduced NAMPT levels in TNBC_{TRAF3IP2KD} cells, they will be transfected with lentiviral vector expressing human recombinant NAMPT to restore expression. We postulate minimal, non-significant metabolic recovery as determined by NAD+/NADH levels, but not reversal of cell senescence and cell cycle arrest. This is because the independent effect of targeting TRAF3IP2 induces mitochondrial injury, thereby irreversibly injuring the cell and decreasing cell proliferation. In a parallel setup, TNBC_{TRAF3IP2KD} and TNBC_{Control} cells treated with nicotinamide mononucleotide (NMN), the reaction product of NAMPT, to determine the biochemical contribution of NAMPT inhibition on NAD+ levels.

### 4. Effect of Targeting TRAF3IP2 on TNBC Cell Senescence

To determine the effect of targeting TRAF3IP2 on SASP, sandwich immunoassay for a panel of pro-inflammatory cytokines (IFN-g, IL-1b, IL-2, IL-6, IL-9, and TNF-a) are performed on conditioned media of TNBC_{TRAF3IP2KD} and compared to TNBC_{Control} cells (V-Plex Proinflammatory Panel 1 Human kit, Mesoscale). In addition, RT-PCR and Western analysis on the panel of cytokines are also performed. To determine whether TNBC_{TRAF3IP2KD} and TNBC_{Control} are in the senescent phase, the classic colorimetric cellular senescence assay detecting senescence-associated β-galactosidase (SA-β-gal) activity are performed (Cellular Senescence Assay, Sigma Aldrich). Apoptosis is assessed by Annexin V staining, in combination with Caspase 3/7 assay system (Promega). In separate experiments, TRAF3IP2 expression are restored in TNBC_{TRAF3IP2 KD} cells using a vector expressing TRAF3IP2 (TNBC_{TRAF3IP2 Restored}), and metabolic reprogramming, cell cycle profile, SASP, and angiogenesis is analyzed as described in corresponding studies.

Severely reduced NAD/NADH levels and decreased NAMPT expression/activation in TNBC_{TRAF3IP2KD} vs. TNBC_{Control} cells are expected. It is theorized that TNBC_{TRAF3IP2KD}, but not TNBC_{Control} cells, will have a significant portion of senescent cells. We anticipate decreased NAMPT activity, and decreased NAD+/NADH ratios in TNBC_{TRAF3IP2KD} vs. TNBC_{Control} cells, signifying metabolic dysfunction. TNBC_{TRAF3IP2KD} but not TNBC_{Control} cells display lower levels and secretion of pro-inflammatory cytokines typical of SASP. The distinction between senescence and apoptosis is important and difficult to dissect, since senescence cells may undergo apoptosis. Because we are working with bulk tumor cells, which include inherently more resistant cells and less resistant cells, targeting TRAF3IP2 may induce apoptosis in the less resistant cells and senescence in the more resistant cells. To account for this, we have included both senescence and apoptosis assays in further study below.

With regards to the experiments that TRAF3IP2 is restored (added back), the effects of TRAF3IP2 on metabolic reprograming, cell proliferation, SASP, NAMPT expression and angiogenesis will be restored in TNBC cells. However, it is possible that the silencing of TRAF3IP2 cause irreversible injury in the TNBC malignant pathways that cannot be reversed by restoration of TRAF3IP2. This latter finding would suggest TRAF3IP2 silencing results in relatively irreversible suppression of key malignant pathways in TNBC cells.

### EXAMPLE 2: DETERMINE THE ROLE OF TRAF3IP2 IN CSC-MEDIATED TUMORIGENESIS AND CHEMORESISTANCE

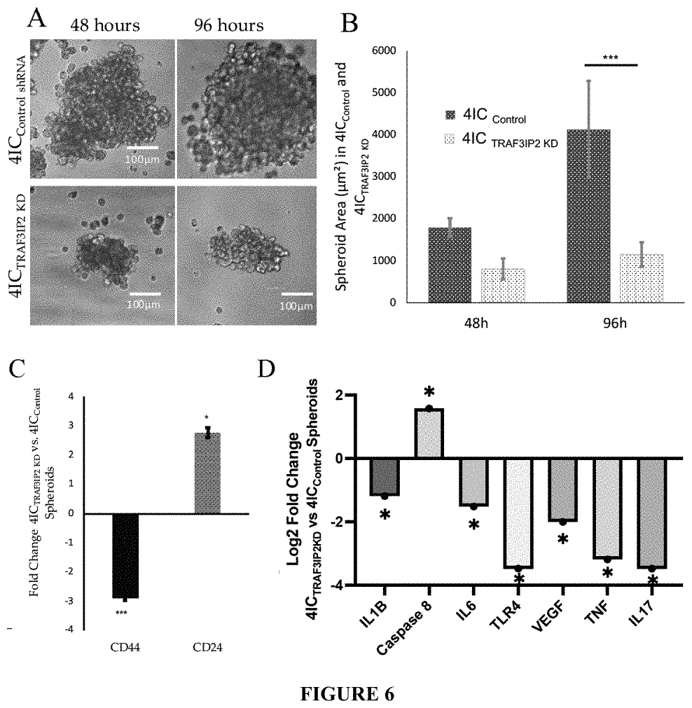

CSCs are largely responsible for tumorigenesis, metastasis, development of chemoresistance and recurrence in TNBC. Neoadjuvant with or without adjuvant chemotherapy remain standard therapeutic regimens in TNBC. In the neoadjuvant setting, chemotherapy serves to debulk the tumor in preparation for surgical resection, while adjuvant chemotherapy aims to eliminate residual cancer post-resection. Neoadjuvant chemotherapy tends to eliminate non-stem bulk tumor cells, while selecting for and enhancing tumor initiating CSCs, which are inherently more resistant to cytotoxic and cytostatic chemotherapy. Likewise, adjuvant therapy is not maximally effective in eliminating CSCs post-resection, as evidenced by chemotherapy refractory and recurrent TNBC disease. Our data show that targeting TRAF3IP2 reduces the spheroid formation ability of TNBC cells indicating reduced CSCs numbers. CSCs are identified by the CD44+/CD24− signature. Targeting TRAF3IP2 significantly reduces CD44 and increased CD24, suggesting suppression of the CSC phenotype (FIG. 6). Also, transcriptomic analysis showed a reduction of inflammatory and angiogenesis markers in CSCs (FIG. 6).

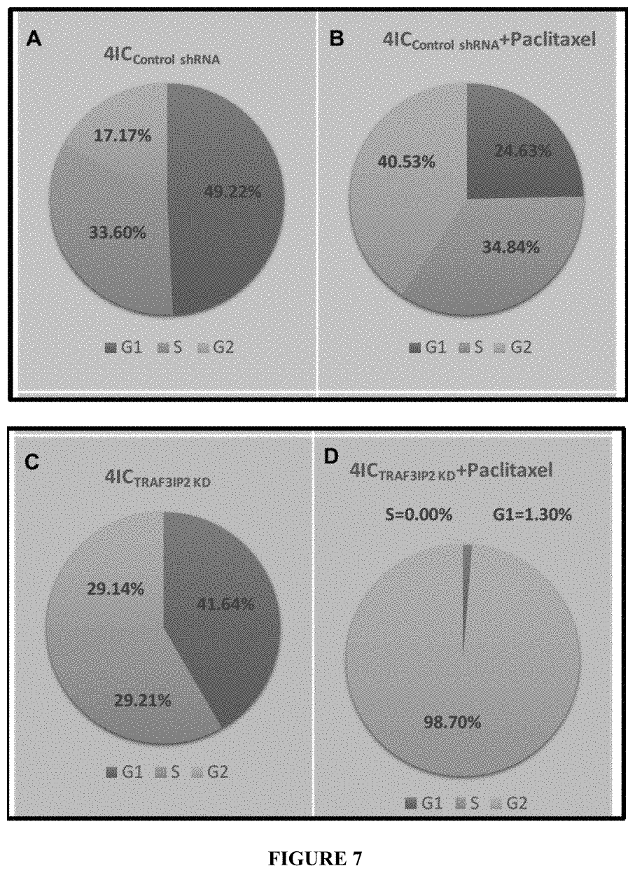

Our current results also show that targeting TRAF3IP2 enhances the efficacy of Paclitaxel in TNBC cells, resulting in suppressed tumor growth. In that experiment, tumor was isolated from a TNBC patient that showed limited response to chemotherapy (4IC PDX), silenced for TRAF3IP2 (4IC_{TRAF3IP2 KD} was done, and cells transduced with scrambled shRNA served as a control, 4IC_{Control shRNA}), treated with Paclitaxel and analyzed for cell cycle profile in vitro. Paclitaxel has been previously shown to arrest cell cycle progression at the G2/M phase, resulting in apoptosis. Our data show that Paclitaxel treatment alone increases the proportion of cells in G2 by 23%, as compared to a 12% increase from TRAF3IP2 knockdown alone. Importantly, targeting TRAF3IP2 reduced the proportion of cells in S phase by 4%. Notably, combining paclitaxel with TRAF3IP2 knockdown completely prevented entry into S phase, and arrested cells completely in the G2/M checkpoint (FIG. 7(A)-(D)).

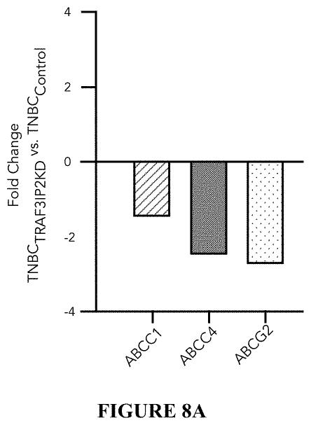

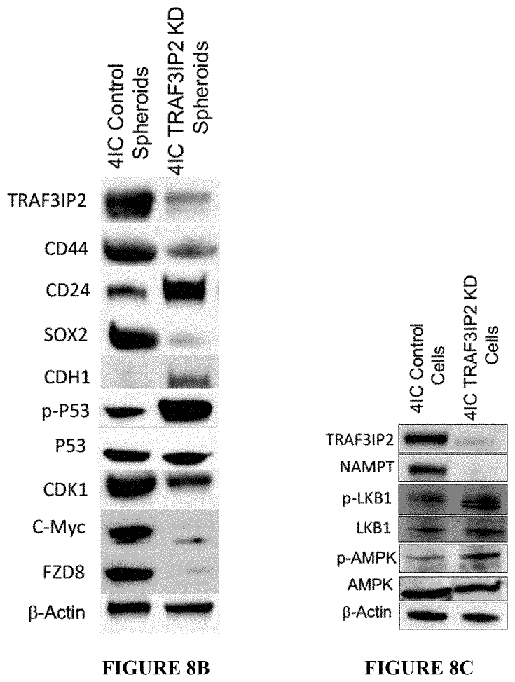

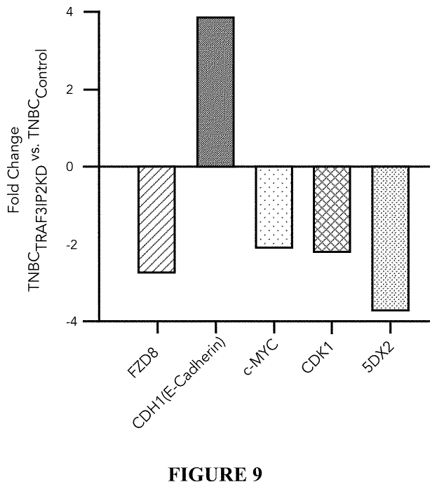

As shown in FIG. 8, the data demonstrate that targeting TRAF3IP2 decreases the expression levels of ABC transporters, which efflux chemotherapeutics and produce chemoresistance. In FIG. 9, we demonstrate that targeting TRAF3IP2 alters Wnt/β-Catenin signaling and reduces Sox2, a critical marker of CSCs. Here, we hypothesize that silencing TRAF3IP2 effectively targets CSCs, and therefore will enhance chemosensitivity to commonly used chemotherapeutics. In Example 2, we will determine the effects of targeting TRAF3IP2 on ABC transporters and chemoresistance to mainstay chemotherapeutics. In Example 2, we also investigate the effect of targeting TRAF3IP2 on Wnt signaling, a major signaling pathway that maintains CSCs. We demonstrate that TRAF3IP2-silenced CSCs have markedly reduced tumorigenicity. We anticipate that these studies will identify targeting TRAF3IP2 as an adjunct treatment enhancer to neoadjuvant and adjuvant chemotherapies, and will significantly reduce CSC enrichment, eliminating disease progression and recurrence.

Bulk TNBC_{TRAF3IP2KD} cells and TNBC_{Control} cells were cultured in serum-free media to isolate the CSCs. Subsequent flow cytometry selecting for CD44⁺/CD24⁻ and ALDH1^{high} was done to confirm the identity of the CSCs. All studies are performed on TNBC_{TRAF3IP2KD} and TNBC_{Control} bulk cells and respective CSCs. The comparison of these groups clearly dissected the effects of targeting TRAF3IP2 on CSCs.

### 5. Effect of TRAF3IP2 on CSCs in TNBC, Silencing TRAF3IP2 Inhibits the Levels and Function of Major ATP Binding Cassette (ABC) Transporters, Implicated in Development of Chemoresistance, in a Panel of TNBC PDXs

ABC cassette transporters utilize ATP to efflux various chemotherapeutic agents from tumor cells, thus generating chemoresistance. Members of the ABC cassette transporter family are distinguished by their substrate specificity (Table 2). Our current data indicate that targeting TRAF3IP2 reduces the expression of key ABC transporters, including ABCG2 (MRP1, multi-drug resistance protein 1), ABCC4, and ABCC1 (FIG. 8A). Additionally, experiments involving Hoechst staining, a substrate of the ABC transporters, demonstrated that TRAF3IP2 silenced TNBC had increased intracellular dye penetration, confirming a decrease in ABC transporter level and/or activity. In addition, targeting TRAF3IP2 reduces mitochondrial function, thus reducing ATP necessary for ABC transporter activity (FIG. 3 and FIG. 8A). These transporters have also been linked to development and maintenance of CSCs in TNBC. Hereby we demonstrate that targeting TRAF3IP2 reduces the levels and activity of ABC transporters, and as such consequently reduces chemoresistance in primarily non-responsive cancer cells.

TABLE 2
Substrates tested to assess the role of
selected ABC transporters on TNBC_{TRAF3IP2KD} and
TNBC_{Control} bulk cells and CSCs

| Transporter | Selected substrates | References |
|---|---|---|
| ABCC1/MRP1 | Anthracyclines, Taxanes, | 50, 51 |
| | Mitoxantrone, Methotrexate | |
| ABCG2/BCRP | 5-Fluorouracil, Methotrexate, | 51 |
| | anthracyclines, irinotecan, | |
| | mitoxantrone | |
| ABCC11/MPR8 | 5-Fluorouracil and Methotrexate | 50, 52 |
| ABCB1/PGP | Anthracyclines, Taxanes, | 53 |
| | Vinca Alkaloids | |

As shown in FIG. 8B, Western blot analysis showed a significant reduction of CD44 and Sox2 levels and increased CD24 cells in 4ICTRAF3IP2KD spheroids. Also, this data showed a significant inhibition of Wnt/β-Catenin signaling by downregulating FZD8 and upregulating p53 and CDH1, as evidenced by downregulation of c-Myc and CDK1 (n=5 independent experiments).

Our current data also indicates that TRAF3IP2 plays a critical role in metabolic reprogramming in TNBC. Silencing TRAF3IP2 inhibits mTOR (Mechanistic Target Of Rapamycin Kinase) and NAMPT (Nicotinamide Phosphoribosyl Transferase), while inducing LKB1 (Liver Kinase B1) and AMPK (AMP-activated Protein Kinase). Shown in FIG. 8C is the Western blot showing down regulation of NAMPT and upregulation of phosphorylated AMPK (p-AMPK) and phosphorylated LKB1 (p-LKB1) in 4ICTRAF3IP2 KD vs. 4ICcontrol shRNA cells, indicating inhibition of cell proliferation (n=5 independent experiments) (*p<0.05, **p<0.01, ***p<0.001).

### 6. Effect of Targeting TRAF3IP2 on ABC Transporters

Expression of ABCC1, ABCG2, ABCC11, and ABCB1 includes analyzing RT-PCR and Western blot on TNBC_{TRAF3IP2KD} and TNBC_{Control} bulk and respective CSCs. Hoechst staining is performed to determine functional status of the ABC transporters (Hoechst 33342 Solution, Thermo Fisher Scientific).

### 7. Effect of Targeting TRAF3IP2 on Chemoresistance

Testing the effect of targeting TRAF3IP2 on enhancing chemosensitivity to paclitaxel, doxorubicin, and cisplatin, three mainstay chemotherapies used in management of TNBC with associated development of chemoresistance. (Table 3). Relevant ranges of concentration responsible for 50% growth inhibitory effect (GI50) of paclitaxel, doxorubicin, cisplatin have previously been reported. TNBC_{TRAF3IP2KD} and TNBC_{Control} bulk and respective CSCs are to be cultured in media (DMEM, 10% FBS for bulk cells or 0% for CSCs, 1% P/S) containing individual chemotherapeutics as detailed in Table 3 for 24, 48, and 72 hours. Cells are harvested by trypsinization and MTT assay are performed to assess cell viability. Further cell cycle analysis utilizing flow cytometry are conducted to reveal cell cycle phase distribution of cells (FxCycle™ Violet Stain, Thermo Fisher Scientific).

TABLE 3
Experimental setting to assess GI50 on
selected chemotherapeutic concentrations on
TNBC_{TRAF3IP2KD} and TNBC_{Control} bulk and CSCs

| Experimental Groups | Low Dose | | Medium Dose | | High Dose | |
|---|---|---|---|---|---|---|
| Paclitaxel | 5 | nM | 12.5 | nM | 20 | nM |
| Doxorubicin | 50 | nM | 100 | nM | 300 | nM |
| Cisplatin | 5 | μM | 25 | μM | 50 | μM |

TNBC_{TRAF3IP2KD} cells generate lower numbers of spheroid CSCs, compared to TNBC_{Control} cells. Based on our data, we still obtain enough CSCs from the TNBC_{TRAF3IP2KD} cells to perform further experiments. However, it is possible that targeting TRAF3IP2 will inhibit the formation of CSCs completely. In this case, we revert to an earlier stock of cells from the same TNBC PDX or include an alternative TNBC PDX (available in the lab). We propose that i) targeting of TRAF3IP2 decreases levels and activity of ABCC1, ABCG2, ABCC11, and ABCB1 in both CSCs and bulk tumor cells. ii) Targeting TRAF3IP2 enhances chemosensitivity at all concentrations used and results in iii) increased cell cycle arrest at G1/S and G2/M cell cycle checkpoints. iv) Targeting TRAF3IP2 in additional other PDXs “difficult to treat” cancer cells has demonstrated similar outcomes.

### 8. Effect of Targeting TRAF3IP2 on Wnt/β-Catenin Signaling

Canonical Wnt signaling activates the β-Catenin/TCF/LEF (T cell factor/lymphoid enhancer factor, respectively) axis resulting in increased breast cancer cell proliferation and maintenance of stemness pathway. The canonical pathway involves binding of a Wnt ligand to the Frizzled receptor (Fzd). Upon binding of Wnt to Fzd and the coreceptor low-density lipoprotein (LRP), β-Catenin is released from intracellular sequestration, and translocates into the nucleus, where it serves as a scaffold protein for TCF and LEF transcription factors. Subsequently, transcription co-activators such as CREB binding protein (CBP) are recruited; the net result is activated transcription of target genes. β-Catenin is sequestered intracellular by at least 2 mechanisms: i) a protein complex consisting of Axin, APC, and the kinases CK1 and GSK3, and ii) the intracellular domain of E-cadherin binds to β-Catenin. In the absence of Wnt, CK1 and GSK3 phosphorylate β-Catenin and result in β-Catenin ubiquitination by E3 TrCP ubiquitin ligase, and subsequent proteasomal degradation. This process is enabled by the presence of Axin, as scaffold protein which forms the protein complex. Upon binding of Wnt to Fzd, GSK3 and CK1 phosphorylate the coreceptor LRP; Axin then binds the phosphorylated LRP and β-Catenin is not degraded; instead, β-Catenin accumulates in the cytoplasm and translocate to the nucleus. In a second mechanism of β-Catenin activation, loss of E-Cadherin releases intracellular β-Catenin, enabling its translocation to the nucleus and pro-transcriptional activity.

Our current data indicate that targeting TRAF3IP2 downregulates Fzd8, a Fzd isoform reported to be selectively increased in TNBCs. Further, Fzd8 is upregulated in TNBCs in response to chemotherapy, suggesting its role in chemoresistance. E-cadherin is a cellular adhesion molecule that forms homotypic interactions between adjacent cells. In breast cancer, downregulation or loss of E-cadherin results in loss of cellular adhesion and enables metastasis. Interestingly, our current data indicate that targeting TRAF3IP2 results in upregulation of E-cadherin (FIG. 9). Therefore, we hypothesize that targeting TRAF3IP2 will inhibit canonical Wnt signaling through downregulation of Fzd8 and upregulation of E-Cadherin, thereby increasing sequestration and proteasomal degradation of β-Catenin. These signaling changes partly explain the decreased tumor cell proliferation and angiogenesis seen in TRAF3IP2 silenced TNBC.

Activation of the Wnt/β-Catenin pathway in TNBC PDX cells (listed in Example 1) are assessed through a TCF/LEF luciferase reporter assay (BPS Bioscience, Inc. San Diego, Calif., USA). To determine the efficacy of TRAF3IP2 knockdown on inhibiting Fzd8/Wnt/β-Catenin activity, TNBC_{TRAF3IP2KD} and TNBC_{SCR} control cells are subjected to two distinct anti-Fzd therapies. Ipafricept (OMP54F28) is a recombinant Fzd8 receptor and human IgG1 Fc fusion protein, which binds to free Wnt ligands in the TME. Therefore, this decoy receptor competes for Wnt binding with the endogenous plasma membrane Fzd8 receptor on the cancer cell, and therefore decreases or inhibits Wnt-driven signaling. In fact, Ipafricept is being studied in several clinical trials as anti-cancer therapy in solid tumors, hepatocellular carcinoma, ovarian cancer, and pancreatic cancers (NCT01608867, NCT02069145, NCT02092363, NCT02050178). In a parallel setup, TNBC_{TRAF3IP2KD} cells and TNBC_{SCR} control cells will be subjected to OMP-18R5 (vanticumab), a monoclonal antibody which targets Fzd1, Fzd2, Fzd5, Fzd7, and Fzd8, and has been shown to prevent tumor growth in solid tumor xenografts, including breast cancer. The study design is summarized in Table 4. Studies in Table 4 will be replicated in two conditions: 1) recombinant Wnt1 will be added to media to stimulate Fzd or 2) no exogenous Wnt1 will be added. This will enable us to dissect the role of non-cancer derived Wnt and cancer cell-derived Wnt on net Wnt signaling.

TABLE 4
Experimental setup for Wnt/β-Catenin signaling studies. Efficacy of targeting TRAF3IP2 on reducing Wnt/β-
Catenin activation compared against clinical drugs OMP-18R5 (monoclonal antibody against FZD8) and
OMP54F28(FZD8 decoy receptor to compete for Wnt binding). Since silencing TRAF3IP2 reduces FZD8
expression and blocks Wnt signaling (FIG. 9), significant reduction in β-Catenin activation and synergy
of combining targeting TRAF3IP2 with the clinical drugs is the assumed reason, Conditions shown are
replicated in two conditions: in the presence (+) or absence (−) of recombinant Wnt.

| | Experiment 1 | | Experimetn 2 | | Experiment 3 | | Experiment 4 | |
|---|---|---|---|---|---|---|---|---|
| Experimental | TNBC- | TNBC | TNBC- | TNBC | TNBC- | TNBC | TNBC- | TNBC |
| setup | _{TRAF3IP2KD} | _{SCR Control} | _{TRAF3IP2KD} | _{SCR Control} | _{TRAF3IP2KD} | _{SCR Control} | _{TRAF3IP2KD} | _{SCR Control} |
| OMP-18R5 | − | − | + | + | − | − | + | + |
| OMP54F28 | − | − | − | − | + | + | + | + |

### 9. Effect of Targeting TRAF3IP2 on E-Cadherin

TNBC_{TRAF3IP2KD} cells and TNBC_{Control} bulk and CSCs are transduced with lentiviral vector carrying shRNA against E-Cadherin or mock. Levels of TRAF3IP2 and E-Cadherin will be determined by RTPCR, Western blot, and immunocytochemistry (ICC). As our current data suggests, TRAF3IP2 knockdown would increase E-Cadherin levels, which will sequester β-Catenin and prevent activation of TCF/LEF.

The loss of E-cadherin would partially restore TCF/LEF activation, supporting the notion that targeting TRAF3IP2 suppresses Wnt signaling partially through upregulation of E-Cadherin. We anticipate the changes will be more robust in the CSC population compared to bulk cells, since Wnt/ β-Catenin is critical to CSC renewal and migration. These results will be compared to TNBCControl cells, where we anticipate a malignant signaling profile: relatively high TRAF3IP2 expression, low E-Cadherin expression, and high Wnt pathway activation. We do not anticipate any major problems. While transduction of cells with two vectors (knockdown of TRAF3IP2 and knockdown of E-Cadherin) is challenging, we have performed this type of experiment before, and have troubleshooted potential problems.

### 10. Effect of Targeting TRAF3IP2 on TNBC Cancer Stem Cells In Vivo: Silencing TRAF3IP2 Inhibits Tumor Growth and Prevents Metastasis In Vivo

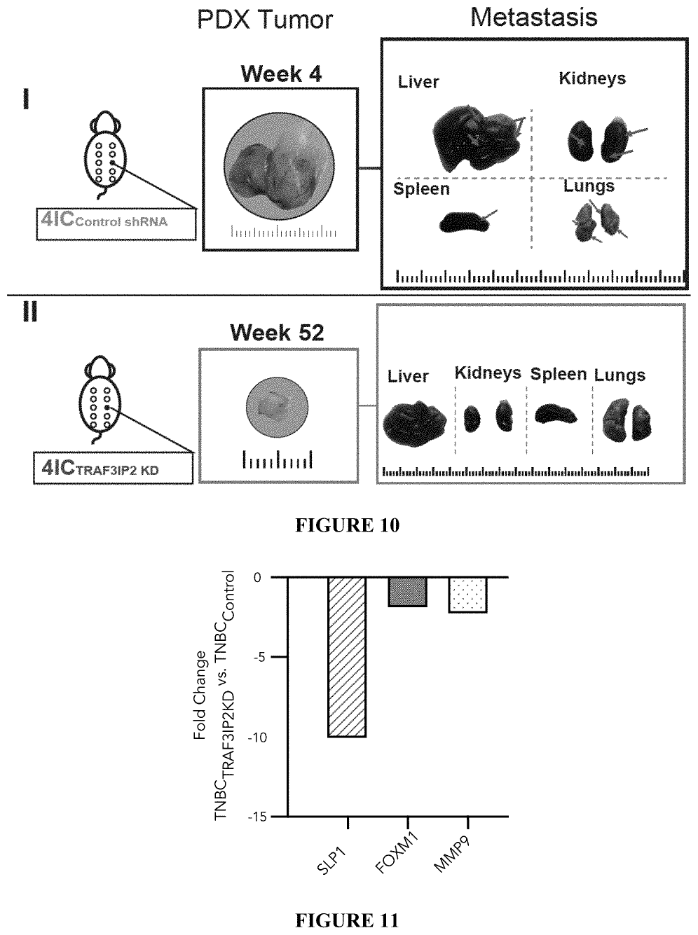

Primary TNBC cell line (4IC) was transduced with lentiviral vector expressing validated TRAF3IP2 shRNA to make 4IC_{TRAF3IP2 KD} cells. Lentiviral vector carrying a non-targeted scrambled shRNA was used to make 4IC_{Control shRNA} cells to serve as a control. Experimental and control tumors were induced in NSG animals (FIG. 10). Analysis showed a markedly decreased tumor weight and volume, and prolonged survival in 4IC_{TRAF3IP2 KD} tumors. Also, no metastases were detected in 4IC_{TRAF3IP2 KD} animals.

TNBC_{TRAF3IP2KD} or TNBC_{Control} bulk or respective CSCs will be injected (1×10⁶ cells in 5 μl PBS) intramammary in female NOD SCID gamma (NSG) mice. Tumor growth will be measured using a caliper. Based on previous experiments, tumors are detectable by caliper by 14 days post-tumor induction. Tumor size, as a primary endpoint, will be measured biweekly for 20 weeks (caliper) and at necropsy. While we showed that silencing TRAF3IP2 reduces CSCs in TNBC cells, we will isolate the limited number of TNBC_{TRAF3IP2KD} CSCs for restoring add back (restoring TRAF3IP2) studies. We will use TNBC_{TRAF3IP2KD} bulk and CSCs for investigations proposed in studies above.

TNBC_{Control} CSCs generate significantly larger tumors than bulk TNBC_{Control}. TNBC_{Control} (bulk and CSCs) will generate significantly larger tumors than TNBC_{TRAF3IP2KD} (bulk and CSCs). It is anticipated that tumorigenesis will be restored in bulk and CSCs of TNBC_{TRAF3IP2} restored-injected animals. It is also possible that the inhibition of CSCs cannot be reversed by restoration of TRAF3IP2. This latter finding would suggest TRAF3IP2 silencing results in relatively irreversible suppression of cell proliferation and CSCs in TNBC cells.

Statistics: Primary endpoint (primary tumor weight): We aim to find a 0.73 g difference between TNBC_{TRAF3IP2KD} or TNBC_{Control} tumor mass. Assuming the same distribution and conditions that led to these findings will hold a 80% power (and alpha=0.05) with n=18/group/PDX. The tumor weight induced by the CSC populations is unknown; however, the difference can be significantly larger than the bulk tumor cells, and therefore since the difference between the means will be larger, a sample size larger than n=18/group/PDX will likely not be necessary. Therefore, for all groups, n=18/group/PDX will be used.

### EXAMPLE 3: TARGETING TRAF3IP2 IN HETEROGENOUS TNBC PDX MODELS ON CELL SURVIVAL BY PREVENTING METASTASIS IN TNBC

Our current data show that targeting TRAF3IP2 in a single PDX model reduces tumor growth and inhibits metastasis in both early and late stages. We also demonstrate that targeting TRAF3IP2 reduces chemoresistance to paclitaxel in a PDX model. We now extend our findings to additional heterogeneous TNBC PDXs: Tu-BcX-4QAN, TU-BcX-49S, and TU-BcX-56S. Additionally, our current data indicates that targeting TRAF3IP2 reduces SLPI (secretory leukocyte peptidase inhibitor) expression by about 10 fold. SLPI has been demonstrated to be a causal factor in TNBC metastasis (FIG. 11). Further, we demonstrate that targeting TRAF3IP2 reduces levels of FOXM1, a transcription factor upregulated in TNBC that drives pro-tumorigenic processes, especially metastasis and invasion. Consistently, MMP9, a target gene of FOXM1 which facilitates extracellular matrix (ECM) degradation and invasion, is downregulated in response to targeting TRAF3IP2. As such we know that independent of TNBC heterogeneity, targeting TRAF3IP2: 1) reduces tumor growth and prevents metastasis in early-stage TNBC, 2) reduces tumor growth and prevents/eliminates metastasis in advanced metastatic TNBC, and 3) reduces TNBC chemoresistance against panel of commonly used chemotherapeutic agents.

### 11. Effect of Targeting TRAF3IP2 on Early Stage TNBC Growth and Metastasis

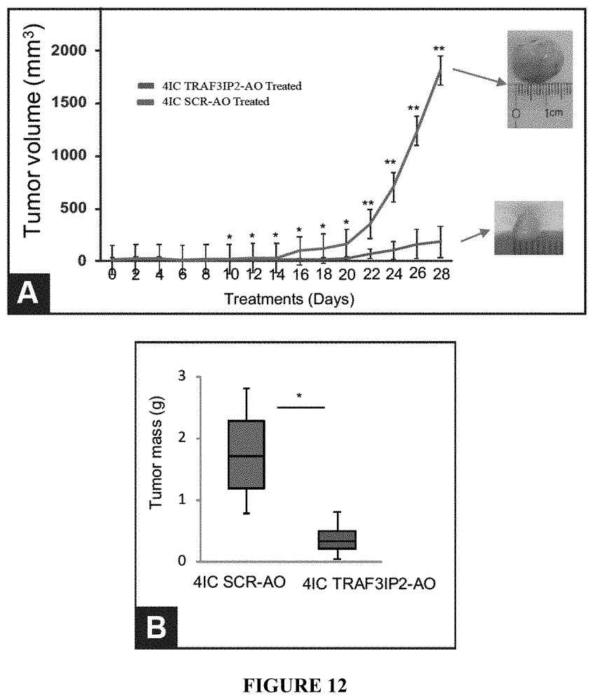

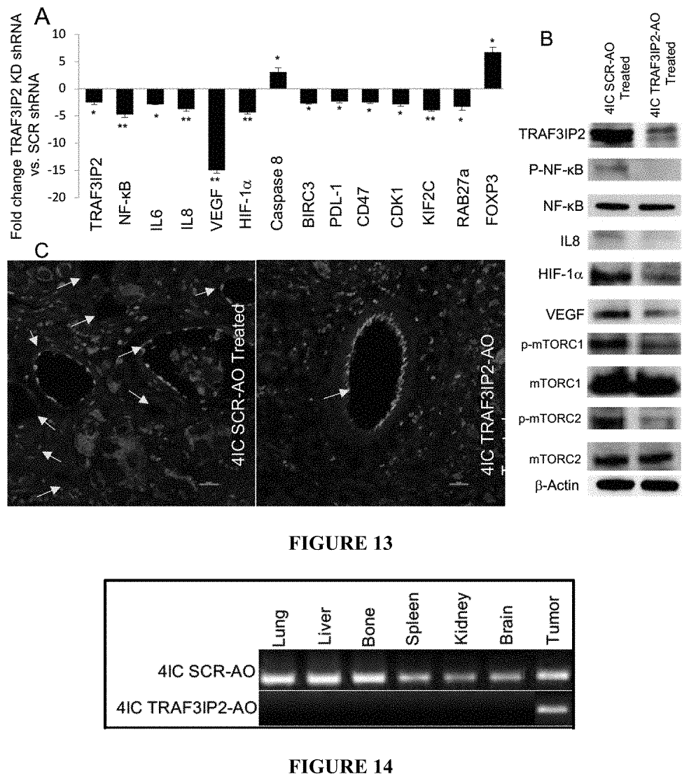

The TU-BcX-4IC (4IC) tumor specimen was used in xenograft studies reported here. The 4IC xenograft tumors were induced by implanting small masses (3 mm dimensions) in mammary fat pad of NSG mice. Treatment regimen was initiated once the implanted tumors were measurable and palpable. As demonstrated in FIG. 12(A), tumors treated with TRAF3IP2-AO showed significantly less growth and smaller volumes and displayed less vascularization compared to control scrambled (SCR)-AO treated mice (p<0.01), demonstrating the efficacy of silencing TRAF3IP2 in suppressing tumor growth. These data indicate that the designed AO is highly specific, and the method of delivery is highly efficient. Post-mortem analysis of residual tumor confirmed that targeting TRAF3IP2 suppresses inflammation, angiogenesis, cell cycle and apoptosis, and genes involved in exosome release (FIG. 13(A)). Protein expression analysis showed that tumors treated with TRAF3IP2-AO displayed significantly lower levels of TRAF3IP2, IL8, IL1b, and VEGF in addition to decreased expression of phosphorylated mTORC1 and mTORC2 in TRAF3IP2-AO treated tumors compared to 4IC SCR-AO treated controls. Together, these molecular data confirm the crucial role of TRAF3IP2 in malignant inflammation and angiogenesis, and strongly suggest the mechanistic involvement of TRAF3IP2 in regulating multiple pro-tumorigenic pathways (FIG. 13(B)). Analysis of residual mass indicated that cells from the host had infiltrated the tumor site forming the greatest majority of the cells (FIG. 13(D)), whereas human breast cancer cells were sparse (negative for human-specific mitochondrial marker; FIG. 13(C)).

Furthermore, TRAF3IP2-AO treated tumors showed a remarkably lower blood vessel density indicated by CD31 staining (an epithelial marker), compared to SCR-AO treated tumors indicating less angiogenesis in TRAF3IP2-AO treated tumors (Arrows in FIG. 13(C)). In addition, PCR to detect human-specific a-satellite DNA sequence of the centromere region of human chromosome 17 indicated no micro-metastasis in the treated group. In contrast, SCR-AO treated mice showed metastasis in lung, liver, bone, kidney, and spleen (FIG. 14).

Tu-BcX-4IC, Tu-BcX-4QAN, TU-BcX-49S, and TU-BcX-56S were luciferase-labeled to enable bioluminescence imaging. After testing multiple antisense oligonucleotides (AO) that specifically target human TRAF3IP2 open reading frame, we identified TRAF3IP2-AO with the highest efficacy and specificity. SCR-AO serves as the control.

Tumor tissue will be implanted in the mammary fat pad of female NOD scid gamma (NSG) mice. Following mammary fat pad implantation of PDX tumors confirmation of tumor induction (−2 weeks) animals will be treated with TRAF3IP2-AO or SCR-AO as control. The AO will be administered subcutaneously in the vicinity of the tumor (150 nm AO every 48 h for 40 days) and animals will be followed for up to 32 weeks. Based on our experience it is anticipated that control SCR-AO-treated animals will need to be euthanized by 6-7 weeks post-tumor induction due to ethical reasons (large tumors); thus, it is expected that most animals followed to 32 weeks will be in the TRAF3IP2-AO treatment group. (ii) The extent of tumor growth will be measured by a digital caliper. Metastasis will be assessed at necropsy (macro-metastasis) and by RT-qPCR to detect human-specific asatellite sequences (micro-metastasis/metastatic niches) in various organs, including lymph nodes, brain, spleen, lung, and liver. Primary tumor weight is the primary endpoint. Overall survival and metastasis are the secondary endpoints.

Primary endpoint (primary tumor volume): Since we do not know how the novel TNBC PDXs behave, we will power the study to detect a significant difference of at least 800mm3 between animals treated with TRAF3IP2-AO and SCR-AO. This is achieved with n=16/group/PDX(alpha=0.05; power=80%).

Secondary endpoint (overall survival): The survival rates of the proposed TNBC PDXs are not known. However, it is our experience that control untreated animals succumb to disease at a median of 45 days, with a maximum standard deviation of 7 days. We will power the study to detect a significant difference of (at least) 7 days between control and treated animals (alpha=0.05; power=80%). This is achieved with n=16/group/PDX, which is consistent with our primary endpoint. Therefore n=16 animals/group/PDX will power our study to detect significance for both the primary and secondary endpoints.

Tertiary endpoint (metastasis): Metastasis will be assessed by gross organ examination, and micro-metastasis is assessed by the method in FIG. 14. SCR-AO treated tumors would exhibit metastasis in lung, liver, bone, spleen, brain, and kidney at the time of necropsy (euthanization at ˜45 days post tumor induction). In our past experience, none of the TRAF3IP2-AO treated tumors had metastases (macro or micro) during the experiment. In order to facilitate the power calculation, each animal will be assigned a metastasis score: 1 point for macrometastasis per organ and 1 point for micrometastasis per organ, for a total of 12 points (see FIG. 14). However, since we do not know how the novel TNBC PDX will behave, we will power our study to detect a significant difference of 2 metastasis points (12 points for SCR-AO, <,=10 points for TRAF3IP2-AO, with a standard deviation of 2 metastasis points (alpha=0.05; power=80%). A n=16 animals/group/PDX is required for this and to maintain consistency with our other endpoints since we will be using the same animals, a final n=16 animals/group/PDX will be used.

### 12. Effect of Targeting TRAF3IP2 in Advanced Metastasis in the PDX Model

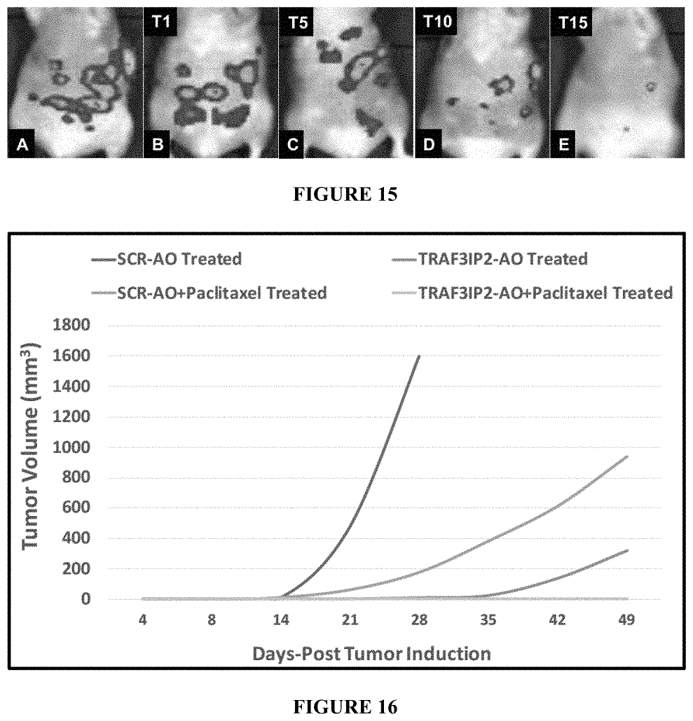

For these experiments, breast tumors were induced by intramammary injection of 1×10⁶ 4IC cells (carrying a luciferase-expressing gene for ‘bioluminescence imaging’) in 5 μl PBS in immunodeficient NIHIII mice (n=5). Treatment of mice was initiated following detection of luciferase signal in secondary sites confirming metastasis (day 42) (FIG. 15(A)). Animals were treated with intraperitoneal injection of TRAF3IP2-AO (100 μg in 50 μl) every 48 hours (FIG. 15(B)-(E)). The results indicate a marked reduction of luciferase signal, reaching undetectable levels after 15 treatments (FIG. 15(E)).

Tumor tissue is implanted as explained in 10 above. Once tumor growth and metastasis (volume>0.1 cm3 and >2 metastatic foci) are detected by imaging, animals are treated with TRAF3IP2-AO using the same methods detailed in item 11 above until metastasis is reduced and eliminated. The primary tumor will be removed once metastasis is confirmed. SCR-AO will serve as a control. The major end points include overall survival and analysis of metastasis. and neo-angiogenesis in residual tumor tissues of TRAF3IP2-AO treated animals and angiogenic markers will be assessed by PCR and IHC.

Primary endpoint (overall survival): The survival rates of the proposed TNBC PDXs are not known. However, it is our experience that control (untreated animals) succumb to disease at median of 45 days, with a maximum standard deviation of 7 days. We will power the study to detect a significant difference of (at least) 7 days between control and treated animals (alpha-0.05; power=80%). This is achieved with n=16/group/PDX.

Secondary endpoint (metastasis): In order to facilitate the power calculation, each animal will be assigned a metastasis score: 1 point for macrometastasis per organ and 1 point for micrometastasis per organ, for a total of 12 points (see FIG. 15). However, since we do not know how the novel TNBC PDX will behave, we will power our study to detect a significant difference of 2 metastasis points (12 points for SCR-AO, <,=10 points for TRAF3IP2-AO, with a standard deviation of 2 metastasis points (alpha=0.05; power=80%). A n=16 animals/group/PDX is required for this. Bioluminesence (FIG. 15) will be used in conjunction to determine metastasis. Bioluminesence readings can be quantified and converted to scores of 0 to 5 (1 is no signal, 5 is maximal signal). The study will be powered to detect a significant difference between SCR-AO (score of 5) and TRAF3IP2-AO (score of 1), with a standard deviation of 2 score points. With an alpha=0.05; power=80%, this is achievable with n=4/group/PDX, and therefore a n=16/group/PDX is more than sufficient to detect a difference based on our expectations.

### 13: Effect of Targeting TRAF3IP2 on TNBC Chemoresistance

Targeting TRAF3IP2 enhances the efficacy of Paclitaxel in TNBC, resulting in maximally suppressed tumor growth. We examined whether the combination therapy (Paclitaxel+TRAF3IP2 knockdown) would be equally effective in vivo. At first, we induced tumors using wild type 4IC cells and a similar set up described in FIG. 12. After tumor induction, each group was treated with one of the following: SCR-AO, SCR-AO and Paclitaxel, TRAF3IP2-AO, or TRAF3IP2-AO and Paclitaxel. Animals in the SCR-AO group had to be euthanized by day 28 due to increased tumor burden. Paclitaxel reduced tumor volume by 42% and TRAF3IP2 targeting reduced tumor size by 78%. Their combination eliminated the tumor (FIG. 16). These clinically relevant in vitro and in vivo data indicate that combining Paclitaxel with TRAF3IP2 knockdown is superior and will overcome resistance of cancer cells to standard chemotherapies.

Following tumor induction (as explained in item 11 above), animals are treated with TRAF3IP2-AO or SCR-AO as control, subcutaneously in the vicinity of the tumor (150 nm AO every 48 h for 40 days). Animals are followed for up to 32 weeks. We test the effects of targeting TRAF3IP2 on chemoresistance using a panel of commonly used chemotherapeutics (all administered intraperitoneal): doxorubicin (10 mg/kg/weekly), cisplatin (10 mg/kg/weekly), cyclophosphamide (50 mg/kg/weekly) according to Table 5.

TABLE 5
Experimental and control groups for Study 12. Animals are induced with primary TNBC. Treatment
will begin upon tumor detection by imaging (~14 days post tumor induction)

| | | | TRAF3IP2- | | | | | |
|---|---|---|---|---|---|---|---|---|
| | | TRAF3IP2- | AO + | TRAF3IP2- | | | | |
| | TRAF3IP2- | AO + | Cyclophos- | AO + | Doxo- | Cyclophos- | | |
| Experiment | AO | Doxorubicine | phamide | Cisplatin | rubicine | phamide | Cisplatin | Saline |
| Control | SCR-AO | SCR-AO + | SCR-AO + | SCR-AO + | Saline | Saline | Saline | Saline |
| | | Doxorubicine | Cyclophos- | Cisplatin | | | | |
| | | | phamide | | | | | |

Based on our results, a significant reduction or complete elimination of metastasis is achievable. While TNBC is highly metastatic, it is possible that some primary TNBCs have an exceedingly high proliferation rate and generate large tumors which necessitate euthanasia at a time point earlier than when maximal Luciferase-detectable macro-metastasis has occurred. In these cases, we will surgically remove the primary tumors and let the metastasis develop to detectable level. Due to the heterogeneity of TNBC, we may see different metastatic outcomes following our initial treatment plan (duration, frequency, and dosage). In such scenarios, such as persistent metastasis, we will adjust the frequency of treatments (for example, increasing the duration, frequency, and dose of treatments). This will give valuable insight into optimization of the treatment strategy of heterogenous TNBCs. Further, our data of complete tumor elimination in animals treated with a combination of TRAF3IP2-AO and paclitaxel

Primary endpoint (tumor size difference between SCR-AO+Chemotherapy and TRAF3IP2-AO): We anticipate finding a 600 mm3 between difference between SCR-AO+Chemotherapy and TRAF3IP2-AO based on FIG. 16. Assuming the same conditions that led to these findings hold, 80% power (and alpha=0.05) would be achieved with n=7/group/PDX.

Secondary endpoint (tumor size difference between TRAF3IP2-AO and TRAF3IP2-AO+Chemotherapy): We anticipate finding approximately 400mm3 difference between TRAF3IP2-AO and TRAF3IP2-AO+Chemotherapy based on FIG. 16. Assuming the same distribution and conditions that led to these findings hold, 80% power (and alpha=0.05) would be achieved with at least n=4/group/PDX. Therefore n=7/group/PDX will be used, enabling the study to achieve statistical significance in both primary and secondary endpoints.

Data have been analyzed with the help of biostatisticians at the Biostatistics Center at Tulane. All data are summarized using descriptive statistics such as mean, median, range, quartile, standard deviation, and standard error. Frequency tables will be drawn up for nominal and ordinal data. Mean values will be compared by ANOVA. Bonferroni's multiple comparison test will be used to compare more than two groups for post-hoc analysis. The categorical data will be analyzed using logistic regression methods. The results will be expressed as odds ratios with the corresponding 95% confidence intervals. The study hypotheses will be tested at 5% significance. Survival studies will be analyzed by Kaplan Meier plots with log rank test, and power analyses for animal groups sizes are included under respective studies. All statistical analyses will be performed using SAS (version 9 or higher in a Windows environment) or R software.

Despite all data being generated based on TNBC, the principle of targeting TRAF3IP2 to reduce the effect of ABC transporters, chemoresistance, metastatic potential, tumor growth, angiogenesis, cell senescence, inflammation, mitochondria oxidative phosphorylation, or metabolic reprogramming is equally applicable to all types of cancers including but not limited to glioblastoma, pancreatic cancer, or metastatic ovarian cancer, because the inventors have shown increased expression of TRAF3IP2 resulting in various cancer progression and signaling pathways as discussed herein.

REFERENCES: the following references are incorporated herein in their entirety for all purposes.

1. C. Blomqvist et al., Influence of treatment schedule on toxicity and efficacy of cyclophosphamide, epirubicin, and fluorouracil in metastatic breast cancer: a randomized trial comparing weekly and every-4-week administration. J Clin Oncol 11, 467-473 (1993).

2. H. Joensuu et al., Combination chemotherapy versus single-agent therapy as first- and second-line treatment in metastatic breast cancer: a prospective randomized trial. J Clin Oncol 16, 3720-3730 (1998).

3. D. Mauri et al., Overall survival benefit for weekly vs. three-weekly taxanes regimens in advanced breast cancer: A meta-analysis. Cancer Treat Rev 36, 69-74 (2010).

4. J. A. Sparano et al., Weekly paclitaxel in the adjuvant treatment of breast cancer. N Engl J Med 358, 1663-1671 (2008).

5. P. Schmid et al., Atezolizumab and Nab-Paclitaxel in Advanced Triple-Negative Breast Cancer. N Engl J Med 379, 2108-2121 (2018).

6. A. Bardia et al., Paclitaxel With Inhibitor of Apoptosis Antagonist, LCL161, for Localized Triple-Negative Breast Cancer, Prospectively Stratified by Gene Signature in a Biomarker-Driven Neoadjuvant Trial. J Clin Oncol, JCO2017748392 (2018).

7. S. Adams et al., Pembrolizumab monotherapy for previously treated metastatic triple-negative breast cancer: cohort A of the phase II KEYNOTE-086 study. Ann Oncol 30, 397-404 (2019).

8. D. Miles et al., Primary results from IMpassion131, a double-blind, placebo-controlled, randomized phase III trial of first-line paclitaxel with or without atezolizumab for unresectable locally advanced/metastatic triple-negative breast cancer. Ann Oncol 32, 994-1004 (2021).

9. L. A. Emens et al., First-line atezolizumab plus nab-paclitaxel for unresectable, locally advanced, or metastatic triple-negative breast cancer: IMpassion130 final overall survival analysis. Ann Oncol 32, 983-993 (2021).

10. M. S. Doyle, E. S. Collins, O. M. FitzGerald, S. R. Pennington, New insight into the functions of the interleukin-17 receptor adaptor protein Act1 in psoriatic arthritis. Arthritis Res Ther 14, 226 (2012).

11. X. Li et al., Act1, an NF-kappa B-activating protein. Proc Natl Acad Sci USA 97, 10489-10493 (2000).

12. F. Lv, L. J. Song, X. H. Wang, F. Qiu, X. F. Li, The role of Act1, a NF-kappaB-activating protein, in IL-6 and IL-8 levels induced by IL-17 stimulation in SW982 cells. Pharm Biol 51, 1444-1450 (2013).

13. N. Manjunath, H. Wu, S. Subramanya, P. Shankar, Lentiviral delivery of short hairpin RNAs. Adv Drug Deliv Rev 61, 732-745 (2009).

14. S. L. Gaffen, R. Jain, A. V. Garg, D. J. Cua, The IL-23-IL-17 immune axis: from mechanisms to therapeutic testing. Nat Rev Immunol 14, 585-600 (2014).

15. N. Bayat et al., Erratum to: Anti-inflammatory Effects of Atorvastatin in Human Glioblastoma Spheroids Cultured in a Three-dimensional Model: Possible Relevance to Glioblastoma Treatment. Mol Neurobiol, (2017).

16. M. Jeon, J. Han, S. J. Nam, J. E. Lee, S. Kim, Elevated IL-1beta expression induces invasiveness of triple negative breast cancer cells and is suppressed by zerumbone. Chem Biol Interact 258, 126-133 (2016).

17. E. U. Alt et al., Targeting TRAF3IP2, Compared to Rab27, is More Effective in Suppressing the Development and Metastasis of Breast Cancer. Sci Rep 10, 8834 (2020).

18. A. Izadpanah et al., Targeting TRAF3IP2 inhibits angiogenesis in glioblastoma. Front Oncol 12, 893820 (2022).

19. E. U. Alt et al., TRAF3IP2, a novel therapeutic target in glioblastoma multiforme. Oncotarget 9, 29772-29788 (2018).

20. L. Yang et al., Low expression of TRAF3IP2-AS1 promotes progression of NONO-TFE3 translocation renal cell carcinoma by stimulating N(6)-methyladenosine of PARP1 mRNA and downregulating PTEN. J Hematol Oncol 14, 46 (2021).

21. C. Luo, X. Pang, IncRNA TRAF3IP2-AS1 Restrains Cervical Cancer Progression by Sponging miR-3677-3p and Acts As a Diagnostic Marker in Cervical Cancer. Crit Rev Eukaryot Gene Expr 33, 43-51 (2022).

22. B. P. Gang, P. J. Dilda, P. J. Hogg, A. C. Blackburn, Targeting of two aspects of metabolism in breast cancer treatment. Cancer Biol Ther 15, 1533-1541 (2014).

23. J. P. Long, X. N. Li, F. Zhang, Targeting metabolism in breast cancer: How far we can go? World J Clin Oncol 7, 122-130 (2016).

24. V. Gouirand, F. Guillaumond, S. Vasseur, Influence of the Tumor Microenvironment on Cancer Cells Metabolic Reprogramming. Front Oncol 8, 117 (2018).

25. S. C. Land, A. R. Tee, Hypoxia-inducible factor 1 alpha is regulated by the mammalian target of rapamycin (mTOR) via an mTOR signaling motif. J Biol Chem 282, 20534-20543 (2007).

26. J. Liang et al., The energy sensing LKB1-AMPK pathway regulates p27(kip1) phosphorylation mediating the decision to enter autophagy or apoptosis. Nat Cell Biol 9, 218-224 (2007).

27. G. Pascual, D. Dominguez, S. A. Benitah, The contributions of cancer cell metabolism to metastasis. Dis Model Mech 11, (2018).

28. G. Gabriely, M. A. Wheeler, M. C. Takenaka, F. J. Quintana, Role of AHR and HIF-1alpha in Glioblastoma Metabolism. Trends Endocrinol Metab 28, 428-436 (2017).

29. M. D. Matossian et al., In-depth characterization of a new patient-derived xenograft model for metaplastic breast carcinoma to identify viable biologic targets and patterns of matrix evolution within rare tumor types. Clin Transl Oncol, (2021).

30. D. Chen et al., Paracrine factors from adipose-mesenchymal stem cells enhance metastatic capacity through Wnt signaling pathway in a colon cancer cell co-culture model. Cancer Cell Int 15, 42-42 (2015).

31. J. Dittmer, B. Leyh, Paracrine effects of stem cells in wound healing and cancer progression (Review). Int J Oncol 44, 1789-1798 (2014).

32. S. Nallanthighal, J. P. Heiserman, D. J. Cheon, The Role of the Extracellular Matrix in Cancer Stemness. Front Cell Dev Biol 7, 86 (2019).

33. F. H. P. S. Lau, S. D., United States Patent: 10344256 - Apparatuses, systems, and methods for culturing cells. . (2019).

34. M. L. Sutherland, K. M. Fabre, D. A. Tagle, The National Institutes of Health Microphysiological Systems Program focuses on a critical challenge in the drug discovery pipeline. Stem Cell Res Ther 4 Suppl 1, I1 (2013).

35. S. D. Scahill, M. Hunt, C. L. Rogers, F. H. Lau, A Microphysiologic Platform for Human Fat: Sandwiched White Adipose Tissue. J Vis Exp, (2018).

36. F. H. Lau et al., Sandwiched White Adipose Tissue: A Microphysiological System of Primary Human Adipose Tissue. Tissue Eng Part C Methods 24, 135-145 (2018).

37. L. D. Shultz et al., Human cancer growth and therapy in immunodeficient mouse models. Cold Spring Harb Protoc 2014, 694-708 (2014).

38. C. J. O'Conor, T. Chen, I. Gonzalez, D. Cao, Y. Peng, Cancer stem cells in triple-negative breast cancer: a potential target and prognostic marker. Biomark Med 12, 813-820 (2018).

39. M. Nedeljkovic, A. Damjanovic, Mechanisms of Chemotherapy Resistance in Triple-Negative Breast Cancer-How We Can Rise to the Challenge. Cells 8, (2019).

40. L. Steward, L. Conant, F. Gao, J. A. Margenthaler, Predictive factors and patterns of recurrence in patients with triple negative breast cancer. Ann Surg Oncol 21, 2165-2171 (2014).

41. J. He et al., Inhibition of USP2 eliminates cancer stem cells and enhances TNBC responsiveness to chemotherapy. Cell Death Dis 10, 285 (2019).

42. K. M. Britton et al., Breast cancer, side population cells and ABCG2 expression. Cancer Lett 323, 97-105 (2012).

43. F. Collina et al., Prognostic Value of Cancer Stem Cells Markers in Triple-Negative Breast Cancer. BioMed research international 2015, 158682 (2015).

44. W. Li et al., Unraveling the roles of CD44/CD24 and ALDH1 as cancer stem cell markers in tumorigenesis and metastasis. Sci Rep 7, 13856 (2017).

45. J. E. Oates et al., Hoechst 33342 side population identification is a conserved and unified mechanism in urological cancers. Stem Cells Dev 18, 1515-1522 (2009).

46. S. J. Isakoff, Triple-negative breast cancer: role of specific chemotherapy agents. Cancer J 16, 53-61 (2010).

47. J. E. Liebmann et al., Cytotoxic studies of paclitaxel (Taxol) in human tumour cell lines. Br J Cancer 68, 1104-1109 (1993).

48. C. J. Lovitt, T. B. Shelper, V. M. Avery, Doxorubicin resistance in breast cancer cells is mediated by extracellular matrix proteins. BMC Cancer 18, 41 (2018).

49. S. Wang et al., Cisplatin suppresses the growth and proliferation of breast and cervical cancer cell lines by inhibiting integrin beta5-mediated glycolysis. Am J Cancer Res 6, 1108-1117 (2016).

50. M. Milanovic et al., Senescence-associated reprogramming promotes cancer stemness. Nature 553, 96- 100 (2018).

51. M. Shipitsin et al., Molecular definition of breast tumor heterogeneity. Cancer Cell 11, 259-273 (2007).

52. X. Xu, M. Zhang, F. Xu, S. Jiang, Wnt signaling in breast cancer: biological mechanisms, challenges and opportunities. Mol Cancer 19, 165 (2020).

53. W. J. Ryu et al., Destabilization of beta-catenin and RAS by targeting the Wnt/beta-catenin pathway as a potential treatment for triple-negative breast cancer. Exp Mol Med 52, 832-842 (2020).

54. T. D. King, M. J. Suto, Y. Li, The Wnt/beta-catenin signaling pathway: a potential therapeutic target in the treatment of triple negative breast cancer. J Cell Biochem 113, 13-18 (2012).

55. J. Xu, J. R. Prosperi, N. Choudhury, O. I. Olopade, K. H. Goss, beta-Catenin is required for the tumorigenic behavior of triple-negative breast cancer cells. PLoS One 10, e0117097 (2015).

56. S. Yin et al., Tumor-initiating cells and FZD8 play a major role in drug resistance in triple-negative breast cancer. Mol Cancer Ther 12, 491-498 (2013).

57. S. Liao, L. Gan, W. Qin, C. Liu, Z. Mei, Inhibition of GSK3 and MEK induced cancer stem cell generation via the Wnt and MEK signaling pathways. Oncol Rep 40, 2005-2013 (2018).

58. H. Clevers, K. M. Loh, R. Nusse, Stem cell signaling. An integral program for tissue renewal and regeneration: Wnt signaling and stem cell control. Science 346, 1248012 (2014).

59. D. R. Pattabiraman, R. A. Weinberg, Targeting the Epithelial-to-Mesenchymal Transition: The Case for Differentiation-Based Therapy. Cold Spring Harb Symp Quant Biol 81, 11-19 (2016).

60. S. A. Mani et al., The epithelial-mesenchymal transition generates cells with properties of stem cells. Cell 133, 704-715 (2008).

61. S. Kochall et al., Isolation of Circulating Tumor Cells in an Orthotopic Mouse Model of Colorectal Cancer. J Vis Exp, (2017).

62. C. Gaspar et al., A targeted constitutive mutation in the APC tumor suppressor gene underlies mammary but not intestinal tumorigenesis. PLoS Genet 5, e1000547 (2009).

63. J. Monteiro et al., Cancer stemness in Wnt-driven mammary tumorigenesis. Carcinogenesis 35, 2-13 (2014).

64. S. Y. Hwang et al., Direct Targeting of beta-Catenin by a Small Molecule Stimulates Proteasomal Degradation and Suppresses Oncogenic Wnt/beta-Catenin Signaling. Cell reports 16, 28-36 (2016).

What is claimed is:

1. A method of treating a subject having cancer, wherein the cancer cells have increased TRAF3IP2 expression, the method comprising the steps of:
a) administering a pharmaceutical composition to the subject, and
b) additionally administering a chemotherapeutic compound to the subject along with the pharmaceutical composition;
c) wherein the pharmaceutical composition comprises an effective amount of at least one targeting sequence against at least a portion of TRAF3IP2 in a pharmaceutically acceptable carrier.

2. The method of claim 1, wherein the targeting sequence against at least a portion of TRAF3IP2 is selected from the group consisting of: a siRNA, a miRNA, a shRNA, an antisense RNA, or an antisense oligonucleotide.

3. The method of claim 2, wherein the targeting sequence is encoded by an expression vector, wherein the targeting sequence is operably coupled to an inducible promoter in the expression vector.

4. The method of claim 1, wherein the cancer is triple negative breast cancer (TNBC), glioblastoma, pancreatic cancer, or metastatic ovarian cancer.

5. The method of claim 1, wherein the chemotherapeutic compound is selected from the group consisting of Actinomycin, All-trans retinoic acid, Azacitidine, Azathioprine, Bleomycin, Bortezomib, Carboplatin, Capecitabine, Cisplatin, Chlorambucil, Cyclophosphamide, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Mechlorethamine, Mercaptopurine, Methotrexate, Mitoxantrone, Oxaliplatin, Paclitaxel, Pemetrexed, Teniposide, Tioguanine, Topotecan, Valrubicin, Vemurafenib, Vinblastine, Vincristine, and Vindesine.

6. The method of claim 1, wherein the subject's metastasis is reduced by at least 50% as compared to the same treatment without administering the pharmaceutical composition.

7. The method of claim 1, wherein the administering step comprises parenteral administration, including injection into a tumor or its metastasis site by transcutaneous, intraarterial, intraductal, intravenous, intradermal, intramuscular, intraperitoneal, or subcutaneous administration.

8. The method of claim 1, wherein Wnt/β-Catenin activation is reduced or inhibited.

9. The method of claim 1, wherein Fzd8 expression in cancer cells is downregulated by at least 50% or inhibited.

10. The method of claim 1, wherein E-Cadherin expression is upregulated by at least 50%.

11. The method of claim 1, wherein tumor growth is inhibited.

12. A composition for treating and preventing metastasis in a subject having a difficult to treat cancer, comprising:
a) a therapeutically effective amount of a TRAF3IP2 silencer; and
b) a pharmaceutically acceptable carrier;
c) one or more anticancer medication, wherein the combination of the TRAF3IP2 silencer and the anticancer medication is at least 20% more effective in treating the cancer than the anticancer medication alone;
d) wherein the TRAF3IP2 silencer comprises at least one targeting sequence against at least a portion of TRAF3IP2 to silence expression of TRAF3IP2 in cancer cells.

13. The composition of claim 12, wherein the anticancer medication is chemotherapeutic agent, immunotherapeutic agent, antiangiogenic agent, or combinations thereof.

14. The composition of claim 12, wherein the cancer is triple negative breast cancer (TNBC), glioblastoma, pancreatic cancer, or metastatic ovarian cancer.

15. A method of sensitizing a tumor prior to treating a patient with the tumor, comprising the steps of:
a) administering a pharmaceutical composition into a subject before at least one of the following procedures for treating the tumor: chemotherapy, radiation therapy, immunotherapy or targeted intervention, wherein the pharmaceutical composition comprises at least one targeting sequence for TRAF3IP2 in a pharmaceutically acceptable carrier in an amount effective for sensitizing the tumor; and
b) performing the procedure for treating the tumor.

16. The method of claim 15, wherein the cancer is triple negative breast cancer, glioblastoma, pancreatic cancer, or metastatic ovarian cancer.
